SEQUENCE LISTING (24 sequences)

SEQ ID NO: 1 - protein, 763 aa, Lactococcus lactis subsp. cremoris
MRFNHFSIVDKNFDEQLAELDQLGFRWSVFWDEKKILKDFLIQSPTDMTVLQANTELDVIEFLKSSIELDWEIFWNITLQLLDFVPNFDFEIGKATEFAKKLNLPQRDVEMTTETIISAFYYLLCSRRKSGMILVEHWVSEGLLPLDNHYHFFNDKSLATFDSSLLEREVVWVESPVDTEQKGKNDLIKIQIIRPKSTEKLPVVITASPYHLGINEKANDLALHEMNVDLEKKDSHKIHVQGKLPQKRPSETKELPIVDKAPYRFTHGWTYSLNDYFLTRGFASIYVAGVGTRGSNGFQTSGDYQQIYSMTAVIDWLNGRTRAYTSRKKTHEIKATWANGKVAMTGKSYLGTMAYGAATTGVDGLEVILAEAGISSWYNYYRENGLVRSPGGFPGEDLDVLAALTYSRNLDGADYLKGNDEYEKRLAEMTTALDRKSGDYNQFWHDRNYLINSDQVRADVLIVHGLQDWNVTPEQAYNFWQALPEGHAKHAFLHRGAHIYMNSWQSIDFSETINAYFSAKLLDRDLNLNLPPVILQENSKEQVWSAVSKFGGDDQLKLPLGKTAVSFAQFDNHYDDESFKKYSKDFNVFKKDLFENKANEAVIDLELPSELTINGPIELEIRLKLNDSKGLLSAQILDFGPKKRLEDKARVKDFKVLDRGRNFMLDDLVELPLVESPYQLVTKGFTNLQNKDLLTVSDLKADEWFTLKFELQPTIYHLEKADKLRVILYSTDFEHTVRDNRKVTYEIDLSQSKLIIPIESVKK

SEQ ID NO: 2 - protein, 182 aa, human rhinovirus 14
GPNTEFALSLLRKNIMTITTSKGEFTGLGIHDRVCVIPTHAQPGDDVLVNGQKIRVKDKYKLVDPENINLELTVLTLDRNEKFRDIRGFISEDLEGVDATLVVHSNNFTNTILEVGPVTMAGLINLSSTPTNRMIRYDYATKTGQCGGVLCATGKIFGIHVGGNGRQGFSAQLKKQYFVEKQ

SEQ ID NO: 3 - protein, 9 aa, Artificial sequence
  Linker 1
GGSGGSGGS

SEQ ID NO: 4 - protein, 9 aa, Artificial sequence
  Linker 2
GSSGSGGSG

SEQ ID NO: 5 - protein, 21 aa, Artificial sequence
  His6 tag
MHHHHHHGGSSGSGSELRTQS

SEQ ID NO: 6 - protein, 242 aa, Artificial sequence
  DsbC based sequence
MDDAAIQQTLAKMGIKSSDIQPAPVAGMKTVLTNSGVLYITDDGKHIIQGPMYDVSGTAPVNVTNKMLLKQLNALEKEMIVYKAPQEKHVITVFTDITCGYCHKLHEQMADYNALGITVRYLAFPRQGLDSDAEKEMKAIWCAKDKNKAFDDVMAGKSVAPASCDVDIADHYVLGVQLGVSGTPAVVLSNGTLVPGYQPPKEMKEFLDEHQKMTSGKGSGSGHHHHHHGGSSGSGSELRTQS

SEQ ID NO: 7 - protein, 174 aa, Artificial sequence
  RL9 based sequence
MKVILLRDVPKIGKKGEIKEVSDGYARNYLIPRGFAKEYTEGLERAIKHEKEIEKRKKEREREESEKILKELKKRTHVVKVKAGEGGKIFGAVTAATVAEEISKTTGLKLDKRWFKLDKPIKELGEYSLEVSLPGGVKDTIKIRVEREEGSGSGHHHHHHGGSSGSGSELRTQS

SEQ ID NO: 8 - protein, 520 aa, Artificial sequence
  NusA based sequence
MNKEILAVVEAVSNEKALPREKIFEALESALATATKKKYEQEIDVRVQIDRKSGDFDTFRRWLVVDEVTQPTKEITLEAARYEDESLNLGDYVEDQIESVTFDRITTQTAKQVIVQKVREAERAMVVDQFREHEGEIITGVVKKVNRDNISLDLGNNAEAVILREDMLPRENFRPGDRVRGVLYSVRPEARGAQLFVTRSKPEMLIELFRIEVPEIGEEVIEIKAAARDPGSRAKIAVKTNDKRIDPVGACVGMRGARVQAVSTELGGERIDIVLWDDNPAQFVINAMAPADVASIVVDEDKHTMDIAVEAGNLAQAIGRNGQNVRLASQLSGWELNVMTVDDLQAKHQAEAHAAIDTFTKYLDIDEDFATVLVEEGFSTLEELAYVPMKELLEIEGLDEPTVEALRERAKNALATIAQAQEESLGDNKPADDLLNLEGVDRDLAFKLAARGVCTLEDLAEQGIDDLADIEGLTDEKAGALIMAARNICWFGDEAGSGSGHHHHHHGGSSGSGSELRTQS

SEQ ID NO: 9 - protein, 396 aa, Artificial sequence
  MBP based sequence
MHHHHHHGSGSGKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTRITKGGSSGSGSELRTQS

SEQ ID NO: 10 - protein, 134 aa, Artificial sequence
  Trx based sequence
MHHHHHHGSGSGSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGGSSGSGSELRTQS

SEQ ID NO: 11 - protein, 181 aa, human rhinovirus 14
GPNTEFALSLLRKNIMTITTSKGEFTGLGIHDRVCVIPTHAQPGDDVLVNGQKIRVKDKYKLVDPENINLELTVLTLDRNEKFRDIRGFISEDLEGVDATLVVHSNNFTNTILEVGPVTMAGLINLSSTPTNRMIRYDYATKTGQCGGVLCATGKIFGIHVGGNGRQGFSAQLKKQYFVEK

SEQ ID NO: 12 - protein, 11 aa, Artificial sequence
  Linker 3
SGSGGSGGSGS

SEQ ID NO: 13 - protein, 21 aa, Artificial sequence
  Fusion partner 1
MHHHHHHGGSSGSGSGSGSGS

SEQ ID NO: 14 - protein, 174 aa, Artificial sequence
  Fusion partner 2
MKVILLRDVPKIGKKGEIKEVSDGYARNYLIPRGFAKEYTEGLERAIKHEKEIEKRKKEREREESEKILKELKKRTHVVKVKAGEGGKIFGAVTAATVAEEISKTTGLKLDKRWFKLDKPIKELGEYSLEVSLPGGVKDTIKIRVEREEGSGSGHHHHHHGGSSGSGSGSGSGS

SEQ ID NO: 15 - protein, 134 aa, Artificial sequence
  Fusion partner 3
MHHHHHHGSGSGSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGGSSGSGSGSGSGS

SEQ ID NO: 16 - protein, 83 aa, Thermotoga maritima
MAHKKSGGVAKNGRDSLPKYLGVKVGDGQIVKAGNILVRQRGTRFYPGKNVGMGRDFTLFALKDGRVKFETKNNKKYVSVYEE

SEQ ID NO: 17 - protein, 13 aa, Artificial sequence
  Linker + HRV14 3C site
SSSGGSEVLFQGP

SEQ ID NO: 18 - protein, 34 aa, Homo sapiens
IKPEAPGEDASPEELNRYYASLRHYLNLVTRQRY

SEQ ID NO: 19 - protein, 29 aa, Homo sapiens
HSQGTFTSDYSKYLDSRRAQDFVQWLMNT

SEQ ID NO: 20 - protein, 31 aa, Artificial sequence
  K34R GLP-1(7-37)
HAEGTFTSDVSSYLEGQAAKEFIAWLVRGRG

SEQ ID NO: 21 - protein, 343 aa, Artificial sequence
  RL9-HRV14 3C sequence
MKVILLRDVPKIGKKGEIKEVSDGYARNYLIPRGFAKEYTEGLERAIKHEKEIEKRKKEREREESEKILKELKKRTHVVKVKAGEGGKIFGAVTAATVAEEISKTTGLKLDKRWFKLDKPIKELGEYSLEVSLPGGVKDTIKIRVEREESSSGSSGSSGSSGPNTEFALSLLRKNIMTITTSKGEFTGLGIHDRVCVIPTHAQPGDDVLVNGQKIRVKDKYKLVDPENINLELTVLTLDRNEKFRDIRGFISEDLEGVDATLVVHSNNFTNTILEVGPVTMAGLINLSSTPTNRMIRYDYATKTGQCGGVLCATGKIFGIHVGGNGRQGFSAQLKKQYFVEKQ

SEQ ID NO: 22 - protein, 14 aa, Artificial sequence
  Linker + HAV site
GGSSGSGSELRTQS

SEQ ID NO: 23 - protein, 182 aa, Coxsackievirus
  MISC_FEATURE (183)..(183): Glutamine at position 183 has been deleted
GPAFEFAVAMMKRNSSTVKTEYGEFTMLGIYDRWAVLPRHAKPGPTILMNDQEVGVLDAKELVDKDGTNLELTLLKLNRNEKFRDIRGFLAKEEVEVNEAVLAINTSKFPNMYIPVGQVTEYGFLNLGGTPTKRMLMYNFPTRAGQCGGVLMSTGKVLGIHVGGNGHQGFSAALLKHYFNDE

SEQ ID NO: 24 - protein, 755 aa, Streptococcus suis
  MISC_FEATURE (212)..(213): QG has been substituted with ET
MRFNQFSFIKKETSVYLQELDTLGFQLIPDASSKTNLETFVRKCHFLTANTDFALSNMIAEWDTDLLTFFQSDRELTDQIFYQVAFQLLGFVPGMDYTDVMDFVEKSNFPIVYGDIIDNLYQLLNTRTKSGNTLIDQLVSDDLIPEDNHYHFFNGKSMATFSTKNLIREVVYVETPVDTAGTGQTDIVKLSILRPHFDGKIPAVITNSPYHETVNDVASDKALHKMEGELAEKQVGTIQVKQASITKLDLDQRNLPVSPATEKLGHITSYSLNDYFLARGFASLHVSGVGTLGSTGYMTSGDYQQVEGYKAVIDWLNGRTKAYTDHTRSLEVKADWANGKVATTGLSYLGTMSNALATTGVDGLEVIIAEAGISSWYDYYRENGLVTSPGGYPGEDLDSLTALTYSKSLQAGDFLRNKAAYEKGLAAERAALDRTSGDYNQYWHDRNYLLHADRVKCEVVFTHGSQDWNVKPIHVWNMFHALPSHIKKHLFFHNGAHVYMNNWQSIDFRESMNALLSQKLLGYENNYQLPTVIWQDNSGEQTWTTLDTFGGENETVLPLGTGSQTVANQYTQEDFERYGKSYSAFHQDLYAGKANQISIELPVTEGLLLNGQVTLKLRVASSVAKGLLSAQLLDKGNKKRLAPIPAPKARLSLDNGRYHAQENLVELPYVEMPQRLVTKGFMNLQNRTDLMTVEEVVPGQWMNLTWKLQPTIYQLKKGDVLELILYTTDFECTVRDNSQWQIHLDLSQSQLILPH

(19) United States
(12) Patent Application Publication
(10) Pub. No.: US 2016/0122793 A1
(43) Pub. Date: May 5, 2016

(54) Fusion Protease

(71) Applicant: NOVO NORDISK A/S, Bagsvaerd (DK)

(72) Inventor: Allan Christian Shaw, Copenhagen N (DK)

(21) Appl. No.: 14/889,993

(22) PCT Filed: May 23, 2014
(86) PCT No.: PCT/EP2014/060696

§ 371 Date: Nov. 9, 2015

Related U.S. Application Data
(60) Provisional application No. 61/834,100, filed on Jun. 12, 2013.

(30) Foreign Application Priority Data
May 24, 2013 (EP) 13169173.5
Jun. 10, 2013 (EP) 13171191.3
Nov. 22, 2013 (EP) 13194053.8

(51) Int. Cl. C12P 21/06; C12N 9/48; C12N 9/50

(52) U.S. Cl. CPC C12P 21/06; C12N 9/506; C12N 9/485

(57) ABSTRACT

This invention relates to novel bifunctional fusion proteases useful for manufacturing a mature protein from a fusion protein. More specifically the present invention relates to bifunctional fusion proteases comprising a picornaviral 3C protease and a Xaa-Pro-dipeptidyl aminopeptidase.

### TECHNICAL FIELD

The present invention relates to the technical fields of protein expression and protein chemistry where a matured protein is to be released from a fusion protein.

### BACKGROUND

Recombinant protein technology allow for the production of large quantities of desirable proteins which may be used for their biological activity. Such proteins are often expressed as recombinant fusion proteins in microbial host cells. The matured protein (protein of interest) is often attached to a fusion partner protein or a smaller amino acid extension in order to increase the expression level, increase the solubility, promote protein folding or to facilitate the purification and downstream processing.

Removal of the fusion partner protein from the fusion protein, to release the mature protein with native N- and C-terminus, may be pivotal for maintaining intact biological activity of the protein as well as for drug regulatory purposes.

Presently a limited number of proteases useful for removal of fusion partner proteins from fusion proteins, which leaves a native N-terminus in the released maturated target protein are available as economically sustainable enzymes for industrial use.

One such enzyme is enterokinase which, however, lacks the specificity to be generally applicable. Other such enzymes are Factor Xa, trypsin, clostripain, thrombin, TEV or rhinoviral 3C protease, all of which either lacks specificity as most proteins comprise internal secondary cleavage sites or leaves an amino acid extension in the C- or N-terminal of the mature protein.

Waugh, Protein Expr. Purif. 80:283-293 (2011) discloses an overview of enzymatic reagents for the removal of affinity tags.

WO92/10576 discloses the use of fusion proteins with DPP IV cleavable extension peptide portions in medicinal preparations.

Xin, Protein Expr. Purif. 2002, 24, pp 530-538 discloses the cloning, expression in Escherichia coli and application of X-prolyl dipeptidyl aminopeptidase from Lactococcus lactis for removal of N-terminal Pro-Pro from recombinant proteins.

Bülow, TIBTECH 9:226-231 (1991) discloses a method for preparation of bi-functional enzymes by gene fusion.

Seo, Appl. Environ. Microbiol. 2000, 66, pp 2484-2490 discloses a bifunctional fusion enzyme of trehalose-6-phosphate synthetase and trehalose-6-phosphate phosphatase.

In the pharmaceutical industry protein pharmaceuticals are now constituting a substantial proportion of the competitive market and efficient processes for the large scale manufacture of these protein pharmaceuticals are therefore needed. A key issue for the industrial use of fusion proteins remains the removal of the fusion protein partner from the fusion protein to liberate the intact matured protein.

Thus, there is a need for an industrial process for specifically removing a fusion partner protein without cleaving internal sites in the mature protein and without leaving any amino acid extension on the mature protein. Preferably this removal of a fusion partner protein is carried out using only a single enzyme which is easily prepared in an industrial process. There is also a need for such a process which can serve this function for many different proteins at mild process conditions in order to prevent unintended chemical and physical changes to the mature protein.

### SUMMARY

It is an object of the present invention to provide a simple, one-step process for providing a matured protein from a fusion protein.

Both picornaviral 3C proteases and Xaa-Pro-dipeptidyl aminopeptidases (XaaProDAP) are very specific enzymes which exhibit complementing activities that have surprisingly been found to be useful for manufacturing of protein pharmaceuticals. However, being proteolytic enzymes they also pose challenges in terms of self-cleavage when fused together as one bifunctional fusion protease.

The combination of the two enzymes in a fusion protease may have the advantage of favourable reaction kinetics due to physical proximity of the two enzymes and thereby also less side-reactions. The combination of the two enzymes in a fusion protease has the further advantage that only one reagent needs to be provided and used. Due to a larger size the fusion protease may also easily be removed from the matured protein by a simple gel-filtration process.

According to a first aspect of the invention there is provided a bifunctional fusion protease comprising the catalytic domains of a picornaviral 3C protease and a XaaProDAP. In one embodiment the bifunctional fusion protease comprises a picornaviral 3C protease and a XaaProDAP.

According to a second aspect of the invention there is provided a bifunctional fusion protease comprising a protein of the formula:

X—Y—Z (I) or

Z—Y—X (II)

wherein
X is a picornaviral 3C protease or a functional variant thereof;
Y is an optional linker;
Z is a Xaa-Pro-dipeptidyl aminopeptidase (XaaProDAP) or a functional variant thereof;
wherein said fusion protease has substantially no self-cleavage activity able to deteriorate at least one of the two proteolytic activities.

In one embodiment the bifunctional fusion protease according to the present invention has the formula (I), i.e. said picornaviral 3C protease or a functional variant thereof is in the N-terminal part of said bifunctional fusion protease.

In another embodiment X is human rhinovirus type 14 3C protease (HRV14 3C) or a functional variant thereof.

In another embodiment Z is an E.C. 3.4.14.11 enzyme or a functional variant thereof.

According to a third aspect of the invention there is provided a method for preparing a bifunctional fusion protease according to the present invention, comprising the recombinant expression of a protein comprising the bifunctional fusion protease in a host cell and subsequently isolating the bifunctional fusion protease.

In one embodiment the method for preparing the bifunctional fusion protease comprises E. coli as said host cell.

According to a fourth aspect of the invention there is provided the use of the bifunctional fusion protease according to the present invention for removing a N-terminal peptide or protein from a larger peptide or protein.

### BRIEF DESCRIPTION OF DRAWINGS

FIG. 1 shows a reducing SDS-PAGE of purified bifunctional HRV14-XaaProDAP fusion protease (Protease 20986). Lane 1: Protein Marker. Numbers indicates size in kDa. Lane 2: Purified Protease 20986.

FIG. 2 shows the deconvoluted mass spectrum of RL27_EVLFQGP_PYY(3-36) following incubation with Protease 20986 for 3 hour at 37° C. using 1:20 molar enzyme to substrate ratio (reaction 1). X-axis: Mass over charge ratio (m/z) in Da. Y-axis: Relative intensity.

FIG. 3 shows the deconvoluted mass spectrum of RL27_EVLFQGP_PYY(3-36) following incubation with Protease 20986 for 3 hour at 37° C. using 1:40 molar enzyme to substrate ratio (reaction 2). X-axis: Mass over charge ratio (m/z) in Da. Y-axis: Relative intensity.

FIG. 4 shows the deconvoluted mass spectrum of RL27_EVLFQGP_PYY(3-36) following incubation with RL9-HRV14 3C protease for 3 hour at 37° C. using 1:20 molar enzyme to substrate ratio (reaction 3). X-axis: Mass over charge ratio (m/z) in Da. Y-axis: Relative intensity.

FIG. 5 shows the deconvoluted mass spectrum of RL27_EVLFQGP_PYY(3-36) following incubation with RL9-HRV14 3C protease for 3 hour at 37° C. using 1:40 molar enzyme to substrate ratio (reaction 4). X-axis: Mass over charge ratio (m/z) in Da. Y-axis: Relative intensity.

FIG. 6 shows the deconvoluted mass spectrum of RL27_EVLFQGP_Glucagon following incubation with Protease 20986 for overnight at 4° C. using 1:500 molar enzyme to substrate ratio (reaction 12). X-axis: Mass over charge ratio (m/z) in Da. Y-axis: Relative intensity.

FIG. 7 shows the deconvoluted mass spectrum of RL27_EVLFQGP_Glucagon following incubation with Protease 28994 overnight at 4° C. using 1:100 molar enzyme to substrate ratio (reaction 13). X-axis: Mass over charge ratio (m/z) in Da. Y-axis: Relative intensity.

FIG. 8 shows the deconvoluted mass spectrum of RL27_EVLFQGP_Glucagon following incubation with Protease 28996 overnight at 4° C. using 1:500 molar enzyme to substrate ratio (reaction 16). X-axis: Mass over charge ratio (m/z) in Da. Y-axis: Relative intensity.

FIG. 9 shows the deconvoluted mass spectrum of RL27_EVLFQGP_Glucagon following incubation with Protease 28997 overnight at 4° C. using 1:500 molar enzyme to substrate ratio (reaction 17). X-axis: Mass over charge ratio (m/z) in Da. Y-axis: Relative intensity.

FIG. 10 shows the deconvoluted mass spectrum of RL27_EVLFQGP_Glucagon following incubation with RL9-HRV14 3C protease overnight at 4° C. using 1:20 molar enzyme to substrate ratio (Reaction 18, control). X-axis: Mass over charge ratio (m/z) in Da. Y-axis: Relative intensity.

FIG. 11 shows the deconvoluted mass spectrum of RL27_EVLFQGP_GLP-1(7-37, K34R) following incubation with Protease 20986 overnight at 4° C. using 1:500 molar enzyme to substrate ratio (reaction 20). X-axis: Mass over charge ratio (m/z) in Da. Y-axis: Relative intensity.

FIG. 12 shows the deconvoluted mass spectrum of RL27_EVLFQGP_GLP-1(7-37, K34R) following incubation with Protease 28994 overnight at 4° C. using 1:100 molar enzyme to substrate ratio (reaction 21). X-axis: Mass over charge ratio (m/z) in Da. Y-axis: Relative intensity.

FIG. 13 shows the deconvoluted mass spectrum of RL27_EVLFQGP_GLP-1(7-37, K34R) following incubation with Protease 28996 overnight at 4° C. using 1:100 molar enzyme to substrate ratio (reaction 23). X-axis: Mass over charge ratio (m/z) in Da. Y-axis: Relative intensity.

FIG. 14 shows the deconvoluted mass spectrum of RL27_EVLFQGP_GLP-1(7-37, K34R) following incubation with Protease 28997 overnight at 4° C. using 1:100 molar enzyme to substrate ratio (reaction 25). X-axis: Mass over charge ratio (m/z) in Da. Y-axis: Relative intensity.

FIG. 15 shows the deconvoluted mass spectrum of RL27_EVLFQGP_GLP-1(7-37, K34R) following incubation with RL9-HRV14 3C protease overnight at 4° C. using 1:20 molar enzyme to substrate ratio (Reaction 27, control). X-axis: Mass over charge ratio (m/z) in Da. Y-axis: Relative intensity.

### DESCRIPTION

According to a first aspect of the invention there is provided a bifunctional fusion enzyme comprising the catalytic domains of a picornaviral 3C protease and a XaaProDAP.

According to a second aspect of the invention there is provided a bifunctional fusion protease comprising a protein of the formula:

X—Y—Z (I) or

Z—Y—X (II)

wherein
X is a picornaviral 3C protease or a functional variant thereof;
Y is an optional linker;
Z is a Xaa-Pro-dipeptidyl aminopeptidase (XaaProDAP) or a functional variant thereof;
wherein said fusion protease has substantially no self-cleavage activity able to deteriorate at least one of the two proteolytic activities.

The method of the invention provides a number of advantages over previously described methods for release of a matured protein from a fusion protein. For example, it has been surprisingly found that a very specific hydrolysis of the fusion protein can be obtained so that the mature protein is released with the correct native N-terminal amino acid in the absence or with a minimum level of related impurities and in high yields. The presence of any related impurities, i.e. proteins resembling the mature protein by having limited differences in chemical structure, is clearly undesirable as they are difficult and thus expensive to remove in a manufacturing process. Additional embodiments have the advantage of allowing release of the matured protein from the fusion protein at reactions conditions having low temperatures.

It has also surprisingly been found that the bifunctional fusion proteases of the present invention can be prepared by recombinant expression in E. coli. Normally it is difficult to express large proteins in E. coli without problems arising. However, the present bifunctional fusion proteases can be prepared by recombinant expression in E. coli, as shown in the disclosed examples of the invention.

The present inventors set out to provide a fusion protease comprising a functional XaaProDAP and a functional picornaviral 3C protease. Such a bifunctional fusion protease should be capable of being expressed in a microorganism, and it should be stable during expression, purification as well as during use for releasing a matured protein from a fusion protein. Multiple technical challenges were encountered during the preparation of the bifunctional fusion protease. Firstly, it was found that the HRV14 3C cleaves itself from a HRV14 3C-XaaProDAP fusion protease, such that the fusion protease was unstable. Secondly, HRV14 3C also cleaves the HRV14 3C-XaaProDAP fusion protease internally in the XaaProDAP from Lactococcus lactis at a site not recognised as a typical HRV14 3C cleavage site. This also rendered the fusion protease unstable. Thirdly, XaaProDAP from Lactococcus lactis may remove dipeptides from the N-terminal of the HRV14 3C-XaaProDAP fusion protease when XaaProDAP is in the C-terminal of the fusion protease. Hence, the first fusion protease exhibited self-cleavage at three different sites resulting in the absence of activity and a challenging task to unravel if expression, purification, catalytic function, stability of the bifunctional fusion protease or a combination of these was the cause.

When designing a bifunctional fusion protease according to the present invention the following steps may be carried out:
a) provide a XaaProDAP or a functional variant which has no QG subsequence accessible on the protein surface,
b) provide a picornaviral 3C protease or a functional variant thereof which, if it is to be in the N-terminal of the bifunctional fusion protease, has no XaaProDAP cleavage site in its N-terminal and has no cleavage site allowing it to excise itself by cleavage at its C-terminal end, and
c) connect the XaaProDAP and the picornaviral 3C protease via an optional amino acid linker sequence such as to constitute a bifunctional fusion protease which can be expressed from a single nucleic acid sequence.

It is to be understood that the terms polypeptide, peptide and protein are used interchangeably in the present context. Also, amino acids are abbreviated according to IUPAC nomenclature as either the single letter or three letter designation.

The bifunctional fusion protease according to the invention preferably exhibits sufficient activity at low temperatures such as from 2-10° C. or from 2-15° C. since this is desirable from an industrial manufacturing viewpoint, e.g. due to control of microbial activities at non-sterile process conditions.

“Xaa-Pro dipeptidyl aminopeptidase” (“XaaProDAP”) as used herein is intended to mean an enzyme having dipeptidase activity specific for Xaa-Pro dipeptides, i.e. the scissile bond connecting the C-terminal of the Xaa-Pro dipeptide with the N-terminal of a peptide or protein of interest. XaaProDAP's are classified according to the international union of Biochemistry and molecular Biology Enzyme (IUBMB) Enzyme Nomenclature as the enzymes EC 3.4.14.11 from the peptidase family S15 and as the enzymes EC 3.4.14.5 from the peptidase family S9B. Non-limiting examples of XaaProDAP are dipeptidyl-peptidase IV (DPP-IV) from mammals. Other non-limiting examples of XaaProDAP are Xaa-Prolyl dipeptidyl aminopeptidase from bacteria such as Lactococcus lactis, Streptococcus thermophilus, Lactobacillus delbrueckii, and Streptococcus suis. Xaa-Prolyl dipeptidyl aminopeptidase from Lactococcus lactis subsp. cremoris CNCM I-1631 has the sequence:

(SEQ ID NO: 1)
MRFNHFSIVDKNFDEQLAELDQLGFRWSVFWDEKKILKDFLIQSPTDM
TVLQANTELDVIEFLKSSIELDWEIFWNITLQLLDFVPNFDFEIGKAT
EFAKKLNLPQRDVEMTTETIISAFYYLLCSRRKSGMILVEHVVVSEGL
LPLDNHYHFFNDKSLATFDSSLLEREVVWVESPVDTEQKGKNDLIKIQ
IIRPKSTEKLPVVITASPYHLGINEKANDLALHEMNVDLEKKDSHKIH
VQGKLPQKRPSETKELPIVDKAPYRFTHGWTYSLNDYFLTRGFASIYV
AGVGTRGSNGFQTSGDYQQIYSMTAVIDWLNGRTRAYTSRKKTHEIKA
TWANGKVAMTGKSYLGTMAYGAATTGVDGLEVILAEAGISSVVYNYYR
ENGLVRSPGGFPGEDLDVLAALTYSRNLDGADYLKGNDEYEKRLAEMT
TALDRKSGDYNQFWHDRNYLINSDQVRADVLIVHGLQDWNVTPEQAYN
FWQALPEGHAKHAFLHRGAHIYMNSWQSIDFSETINAYFSAKLLDRDL
NLNLPPVILQENSKEQVWSAVSKFGGDDQLKLPLGKTAVSFAQFDNHY
DDESFKKYSKDFNVFKKDLFENKANEAVIDLELPSELTINGPIELEIR
LKLNDSKGLLSAQILDFGPKKRLEDKARVKDFKVLDRGRNFMLDDLVE
LPLVESPYQLVTKGFTNLQNKDLLTVSDLKADEWFTLKFELQPTIYHL
EKADKLRVILYSTDFEHTVRDNRKVTYEIDLSQSKLIIPIESVKK

The XaaProDAP may be an enzyme naturally occurring in e.g. bacteria or mammals, but it may also be a functional variant of such an enzyme. A non-limiting example of a functional variant is an analogue, an extended or a truncated version of a naturally occurring XaaProDAP which functional variant retain dipeptidase activity specific for Xaa-Pro dipeptides.

The picornaviral 3C proteases (or Protein 3C, Picornian 3C or Picornaviral 3C) are a group of cysteine proteases with a serine proteinase-like fold that are responsible for generating mature viral proteins from a precursor polyprotein in vira from the Picornaviridae family.

“Picornaviral 3C protease” as used herein is intended to mean a protease originating from the family Picorna viridae including functional variants thereof, which protease cleave the peptide bond between a P1-P1′ Gln-Gly pair where the scissile bond connects Gln and Gly (where P1 and P1′ according to commonly used notation denote the first amino acids on the N-terminal and C-terminal sides of the scissile bond, respectively). Several picornaviral 3C proteases, have an additional preference for Pro in P2′ where P2′ denote the second amino acid on the C-terminal side of the scissile bond. Enzymes with this substrate specificity are typically isolated from virus of the genus enterovirus, which currently comprises Coxsackie virus, Echovirus, Enterovirus, Poliovirus and Rhinovirus. Non-limiting examples of such picornaviral 3C proteases are Human Rhino Virus type 14 3C (HRV14 3C) protease having the sequence GPNTEFALSLLRKNIMTITTSKGEFTGLGIHDRVCVIPTHAQPGDDVLVNGQKIRVKDKYKLV DPENINLELTVLTLDRNEKFRDIRGFISEDLEGVDATLVVHSNNFTNTILEVGPVTMAGLINLS STPTNRMIRYDYATKTGQCGGVLCATGKIFGIHVGGNGRQGFSAQLKKQYFVEKQ (SEQ ID NO: 2), Enterovirus 71 3C protease, Coxsackievirus A16 3C protease, Coxsackievirus B3 3C protease, cowpea mosaic comovirus-type picornain 3C and Human Poliovirus 3C protease. These 3C proteases are able to release a protein with Gly-Pro in the N-terminal from a large fusion protein and can often be identified by having a Gly-Pro naturally occurring in their own native N-terminal. According to the present invention the picornaviral 3C protease may be an enzyme naturally occurring in the Picorna viridae, but it may also be a functional variant of such an enzyme. A non-limiting example of a functional variant is an analogue, an extended or a truncated version of a naturally occurring picornaviral 3C protease which functional variant retain substrate specificity for the Gln-Gly pair.

“Substantially no self-cleavage activity able to deteriorate at least one of the two proteolytic activities” as used herein is intended to mean that the bifunctional fusion protease under expression conditions, purification conditions, storage conditions and manufacturing use for cleaving precursors for a target protein, does not cleave itself or does only cleave itself at a very slow rate which does not prevent its intended use for cleaving precursors for a target protein.

In one embodiment, the “substantially no self-cleavage activity able to deteriorate at least one of the two proteolytic activities” is determined by the bifunctional fusion protease under manufacturing conditions being sufficiently stable for cleaving a precursor for a target protein.

In another embodiment the determination of said fusion protease having substantially no self-cleavage activity able to deteriorate at least one of the two proteolytic activities is determined by said bifunctional fusion protease being suitable for the intended use thereof.

In another embodiment the determination of said fusion protease having substantially no self-cleavage activity able to deteriorate at least one of the two proteolytic activities is determined by at least 50% of the bifunctional fusion protease being intact after incubating said bifunctional fusion protease at a concentration of 0.5 mg/mL, in 1×PBS buffer, pH 7.4 at the temperature 37° C. for 3 hours.

In another embodiment the determination of said fusion protease having substantially no self-cleavage activity able to deteriorate at least one of the two proteolytic activities is determined by at least 50% of both the picornaviral 3C protease activity and the XaaProDAP activity of the bifunctional fusion protease being intact after incubating said bifunctional fusion protease at a concentration of 0.5 mg/mL, in 1×PBS buffer, pH 7.4 at the temperature 37° C. for 3 hours.

In another embodiment the determination of said fusion protease having substantially no self-cleavage activity able to deteriorate at least one of the two proteolytic activities is determined by at least 80% of both the picornaviral 3C protease activity and the XaaProDAP activity of the bifunctional fusion protease being intact after incubating said bifunctional fusion protease at a concentration of 0.5 mg/mL, in 1×PBS buffer, pH 7.4 at the temperature 37° C. for 3 hours.

In another embodiment the determination of said fusion protease having substantially no self-cleavage activity able to deteriorate at least one of the two proteolytic activities is determined by at least 50% of both the picornaviral 3C protease activity and the XaaProDAP activity of the bifunctional fusion protease being intact after incubating said bifunctional fusion protease at a concentration of 0.5 mg/mL, in 1×PBS buffer, pH 7.4 at the temperature 4° C. for 24 hours.

In another embodiment the determination of said fusion protease having substantially no self-cleavage activity able to deteriorate at least one of the two proteolytic activities is determined by at least 80% of both the picornaviral 3C protease activity and the XaaProDAP activity of the bifunctional fusion protease being intact after incubating said bifunctional fusion protease at a concentration of 0.5 mg/mL, 1×PBS buffer, pH 7.4 at the temperature 4° C. for 24 hours. “Matured protein” as used herein is intended to mean a protein, a peptide or a polypeptides of interest, or an extended version thereof which extended version can be cleaved at its N-terminus by XaaProDAP. The matured protein is often present as a fusion protein during its manufacture, such as a protein comprising a tag sequence, an optional linker sequence, and a picornaviral 3C protease site in addition to the matured protein. Non-limiting examples of a mature protein is glucagon, PYY(3-36), GLP-1(7-37), Arg34-GLP1(7-37), Arg34-GLP-1(9-37) and Arg34-GLP-1(11-37). Using the commonly used single letter abbreviation of amino acid residues, for instance, Arg34-GLP-1(7-37) is K34R-GLP-1(7-37) (also designated as GLP-1(7-37, K34R)).

“Fusion protein” as used herein is intended to mean a hybrid protein which can be expressed by a nucleic acid molecule comprising nucleotide sequences encoding at least two different proteins. For example, a fusion protein can comprise a tag protein fused with a protein having an activity of pharmaceutical interest. Fusion proteins are often used for improving recombinant expression of therapeutic proteins as well as for improved recovery and purification of such proteins from cell cultures and the like. Fusion proteins may also be used to combine two different enzyme activities into a single protein. Fusion proteins may also comprise artificial sequences, e.g. a linker sequence.

“Fusion protease” as used herein is intended to mean a hybrid protein which can be expressed by a nucleic acid molecule comprising nucleotide sequences encoding at least two different proteins which both have proteolytic activity. For example, a fusion protease can comprise two different proteases, e.g. an endopeptidase and an exoprotease. A fusion protease can also comprise e.g. a tag protein fused to the two proteolytic proteins.

In one embodiment, the two different proteins comprised by the fusion protease exhibit two different proteolytic activities. In another embodiment, the two different proteins comprised by the fusion protease are proteases or functional variants thereof which are originating from different organisms.

XaaProDAP proteases have a protein structure comprising two. alpha helixes linked together via a large protein loop. This loop is exposed at the surface of the protein and thus is susceptible to cleavage by a picornaviral 3C protease, in particular when this picornaviral 3C protease and the XaaProDAP are comprised in a bifunctional fusion protease. The loop connecting the two small alpha-helices of XaaProDAP represents a highly conserved region among XaaProDAP proteases. In SEQ ID NO:1 the loop is the subsequence spanning from residue approximately 223 to 270. The present inventor found that the XaaProDAP was unstable when fused to HRV14 3C and that this was caused by HRV14 3C cleaving at the QG subsequence at positions 241-242. This was highly surprising as the loop does not comprise a subsequence which is a common picornaviral 3C protease cleavage site. Hence, this particular challenge was solved by using a XaaProDAP functional variant which had the QG amino acids substituted for other amino acids, e.g. ET.

“Fusion partner protein” or “fusion partner” as used herein is intended to mean a protein which is part of a fusion protein, i.e. one of the at least two proteins encompassed by the fusion protein. Non-limiting examples of fusion partner proteins are tag proteins and solubilisation domains such as His6-tags, Maltose-binding protein, Thioredoxin, etc.

“Fusion enzyme” as used herein is intended to mean a fusion protein comprising at least two proteins which are both enzymes (in the sense that the two proteins have backbone sequences that are covalently connected).

“Tag protein” or “tag” as used herein is intended to mean a protein which is attached to another protein in order to facilitate or improve the manufacture of said other protein, e.g. facilitating or improving the recombinant expression, recovery and/or purification of said other protein. Non-limiting examples of tag proteins are His6-tags, Glutathione S-transferase (GST), Maltose-binding Protein (MBP), Staphylococcus aureus protein A, biotinylated peptides and highly basic proteins from thermophilic bacteria as described in WO2006/108826 and WO2008/043847.

“Tag sequence” as used herein is intended to mean a sequence comprising a protein. A tag sequence may optionally also comprise an additional sequence, e.g. a linker sequence. Protein tags are peptide sequences genetically grafted onto a recombinant protein, which may be removable by chemical agents or by enzymatic means, such as proteolysis. Tags are attached to proteins for various purposes, such as to facilitate expression or secretion from a cell, to increase solubility or to facilitate proper folding of the protein.

“Linker” as used herein is intended to mean an amino acid sequence which is typically used to facilitate the function, folding or expression of fusion proteins. It is known to persons skilled in the art that two proteins present in the form of a fusion enzyme may interfere with the enzyme activities of each other, an interaction that can often be eliminated or reduced by the insertion of a linker between the two enzyme sequences.

“Analogues” as used herein is intended to mean proteins which are derived from another protein by means of substitution, deletion and/or addition of one or more amino acid residues from the protein. Non-limiting example of analogues of GLP-1(7-37) are K34R-GLP-1(7-37) where residue 34 has been substituted by an arginine residue and K34R-GLP-1(9-37) where residue 34 has been substituted with an arginine residue and amino acid residues 7-8 have been deleted (using the common numbering of amino acid residues for GLP-1 peptides).

“Functional variant” as used herein is intended to mean a chemical variant of a certain protein which has an altered sequence of amino acids but retains substantially the same function as the original protein. Hence a functional variant is typically a modified version of a protein wherein as few modifications are introduced as necessary for the modified protein to obtain some desirable property while preserving substantially the same function as the original protein. Non-limiting examples of functional variants are e.g. extended proteins, truncated proteins, fusion proteins and analogues. Non-limiting examples of functional variants of HRV14 3C are e.g. His6 tagged HRV14 3C, GST-tagged HRV14 3C and HRV14 3C truncated such as not to include the N-terminal GP dipeptide. Non-limiting functional variants of GLP-1(7-37) are K34R-GLP-1(7-37).

In one embodiment, a function variant of a protein comprises from 1-2 amino acid substitutions, deletions or additions as compared said protein. In another embodiment, a functional variant comprises from 1-5 amino acid substitutions, deletions or additions as compared to said protein. In another embodiment, a functional variant comprises from 1-15 amino acid substitutions, deletions or additions relative to the corresponding naturally occurring protein or naturally occurring sub-sequence of a protein.

A “Solubilisation domain” as used herein is intended to mean a protein which is part of a fusion protein and which is to render said fusion protein more soluble than the protein of interest itself under certain conditions. Non-limiting examples of solubilisation domains are DsbC (Thiol:disulfide interchange protein), RL9 (Ribosomal Protein L9) as described in WO2008/043847, MPB (Maltose-binding Protein), NusA (Transcription termination/antitermination protein) and Trx (Thioredoxin).

The term “enzymatic treatment” as used herein is intended to mean a contacting of a substrate protein with an enzyme which catalyses at least one reaction involving said substrate protein. One common enzymatic treatment is the contacting of a fusion protein with an enzyme having proteolytic activity in order to separate two proteins being constituents of the fusion protein.

According to a fourth aspect of the invention there is provided the use of the bifunctional fusion protease according to the present invention for removing an N-terminal peptide or protein from a larger peptide or protein to obtain a mature protein with the intended N-terminal aa residue. Said larger peptide or protein typically is a fusion protein comprising a matured protein and one or more tag sequences serving to facilitate recombinant expression, proper folding of the protein, purification purposes, etc.

In one embodiment, said larger peptide or protein is contacted with said bifunctional fusion protease under suitable reaction conditions and for sufficient time to liberate the majority of said N-terminal peptide. The reaction conditions may for instance include a pH in the range from about 6.0 to about 9.0, in the range from about 7.0 to about 8.5, in the range from about 7.5 to about 8.5, in the range from about 8.0 to about 9.0, or in the range from about 6.0 to about 7.0. The reaction condition may include a temperature in the range from about 0° C. to about 50° C., in the range from about 30° C. to about 37° C., in the range from about 0° C. to about 15° C., in the range from about 0° C. to about 10° C., in the range from about 2° C. to about 10° C., in the range from about 5° C. to about 15° C., in the range from about 0° C. to about 5° C., or in the range from about 2° C. to about 8° C. In another embodiment the reaction condition include a pH in the range from about pH 7.5 to about pH 8.5 and a temperature in the range from about 4° C. to about 10° C. In a yet further embodiment the reaction conditions include a reaction time in the range from about one minute to about 3 hours. In yet another embodiment the reaction conditions include a reaction time in the range from about 3 hours to about 24 hours. In yet another embodiment the reaction time is in the range from about 3 hours to about 24 hours, in the range from about 3 hours to about 16 hours, in the range from about 6 hours to about 24 hours, in the range from about 10 hours to about 16 hours, In another embodiment the reaction conditions include an aqueous medium comprising phosphate buffered saline, such as 50 mM sodium phosphate plus 0.9% sodium chloride. Phosphate buffered saline (abbreviated PBS) is a buffer solution commonly used and typically is a water-based salt solution containing sodium phosphate, sodium chloride and, in some solutions, potassium chloride and potassium phosphate. A typical 1×PBS buffer used for enzymatic reactions in the present invention is (8.05 mM Na2HPO4x2H2O, 1.96 mM KH2PO4, 140 mM NaCl, pH 7.4).

Other useful buffers for the reaction medium may be TRIS (tris(hydroxymethyl)-aminomethane) or HEPES (4-(2-hydroxyethyl)-1-piperazineethanesulfonic acid) buffers.

In another embodiment, the bifunctional fusion protease is co-expressed with said larger peptide or protein to release the protein of interest in vivo during expression in a host cell. In another embodiment said larger peptide or protein is contacted with said bifunctional fusion protease following isolation of these two proteins from the host cells used for their expression.

In another embodiment said larger peptide or protein is selected from peptides or proteins comprising a peptide selected from GLP-1 (Glucagon-like peptide 1), glucagon, Peptide YY (PYY), amylin and functional variants thereof.

In yet another embodiment said larger peptide or protein has a size of less than 200 amino acid residues, less than 150 amino acid residues, less than 100 residues, or less than 60 amino acid residues.

“Application” means a sample containing the fusion protein which is loaded on a purification column.

“Flow through” means the part of the application containing host cell proteins and contaminants which do not bind to the purification column

“Main peak” refers to the peak in a purification chromatogram which has the highest UV intensity and which contains the fusion protein

“UV 280 intensity” is the absorbance at a wavelength of 280 nm at which proteins will absorb, measured in milliabsorbance units

“UV215” is the absorbance at a wavelength of 215 nm at which proteins will absorb, measured in milliabsorbance units

“IPTG” is isopropyl-β-D-thiogalactopyranoside.

TIC is Total Ion Count

HPLC is high performance liquid chromatography

LC-MS refers to liquid chromatography mass spectrometry.

“% Purity” is defined as the amount of a specific protein divided by the amount of specific protein+the amount of contaminants×100

SDS-PAGE is sodium dodecyl sulfate polyacrylamide gel electrophoreses

According to a third aspect of the invention there is provided a method for preparing a bifunctional fusion protease according to the present invention, comprising the recombinant expression of a protein comprising the bifunctional fusion protease in a host cell and subsequently isolating the bifunctional fusion protease.

In one embodiment the method for preparing the bifunctional fusion protease comprises E. coli as said host cell.

In another embodiment the method for preparing the bifunctional fusion protease comprises the isolation of said bifunctional fusion protease as a soluble protein.

In another embodiment the method for preparing the bifunctional fusion protease comprises the isolation of said bifunctional fusion protease as a soluble protein without the use of a refolding step.

In another embodiment the method for preparing the bifunctional fusion protease comprises a bifunctional fusion protease having the formula (I) as depicted in embodiment 2, i.e. said picornaviral 3C protease or a functional variant thereof is in the N-terminal part of said bifunctional fusion protease.

The bifunctional fusion protease may be produced by means of recombinant protein technology. In general, cloned wild-type picornian 3C protease and cloned wild-type XaaProDAP nucleic acid sequences or functional variants thereof are modified to encode the desired fusion protein. This modification includes the in-frame fusion of the nucleic acid sequences encoding the two or more proteins to be expressed as a fusion protein. Such a fusion protein can be the bifunctional fusion protease, with or without a linker peptide, as well as the bifunctional fusion protease fused to a tag, e.g. a His-tag or a solubilization domain (such as DsbC, RL9, MBP, NusA or Trx). This modified sequence is then inserted into an expression vector, which is in turn transformed or transfected into the expression host cells.

The nucleic acid construct encoding the bifunctional fusion protease may suitably be of genomic, cDNA or synthetic origin. Amino acid sequence alterations are accomplished by modification of the genetic code by well known techniques.

The DNA sequence encoding the bifunctional fusion protease is usually inserted into a recombinant vector which may be any vector, which may conveniently be subjected to recombinant DNA procedures, and the choice of vector will often depend on the host cell into which it is to be introduced. Thus, the vector may be an autonomously replicating vector, i.e. a vector, which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

The vector is preferably an expression vector in which the DNA sequence encoding the bifunctional fusion protease is operably linked to additional segments required for transcription of the DNA. The term, “operably linked” indicates that the segments are arranged so that they function in concert for their intended purposes, e.g. transcription initiates in a promoter and proceeds through the DNA sequence coding for the polypeptide until it terminates within a terminator.

Thus, expression vectors for use in expressing the bifunctional fusion protease will comprise a promoter capable of initiating and directing the transcription of a cloned gene or cDNA. The promoter may be any DNA sequence, which shows transcriptional activity in the host cell of choice and may be derived from genes encoding proteins either homologous or heterologous to the host cell.

Additionally, expression vectors for expression of the bifunctional fusion protease will also comprise a terminator sequence, a sequence recognized by a host cell to terminate transcription. The terminator sequence is operably linked to the 3′ terminus of the nucleic acid sequence encoding the polypeptide. Any terminator which is functional in the host cell of choice may be used in the present invention.

Expression of the bifunctional fusion protease can be aimed for either intracellular expression in the cytosol of the host cell or be directed into the secretory pathway for extracellular expression into the growth medium.

Intracellular expression is the default pathway and requires an expression vector with a DNA sequence comprising a promoter followed by the DNA sequence encoding the bifunctional fusion protease polypeptide followed by a terminator.

To direct the bifunctional fusion protease into the secretory pathway of the host cells, a secretory signal sequence (also known as signal peptide or a pre sequence) is needed as an N-terminal extension of the bifunctional fusion protease. A DNA sequence encoding the signal peptide is joined to the 5′ end of the DNA sequence encoding the bifunctional fusion protease in the correct reading frame. The signal peptide may be that normally associated with the protein or may be from a gene encoding another secreted protein.

The procedures used to ligate the DNA sequences coding for the bifunctional fusion protease, the promoter, the terminator and/or secretory signal sequence, respectively, and to insert them into suitable vectors containing the information necessary for replication, are well known to persons skilled in the art (cf., for instance, Sambrook et al., Molecular Cloning: A Laboratory Manual, Cold Spring Harbor, N. Y., 1989).

The host cell into which the DNA sequence encoding the bifunctional fusion protease is introduced may be any cell that is capable of expressing the bifunctional fusion protease either intracellularly or extracellularly. If posttranslational modifications are needed, suitable host cells include yeast, fungi, insects and higher eukaryotic cells such as mammalian cells.

### Bacterial Expression

Examples of suitable promoters for directing the transcription of the nucleic acid constructs in a bacterial host cell are, for expression in E. coli, the promoters obtained from the lac operon, the trp operon and hybrids thereof trc and tac, all from E. coli (DeBoer et al., 1983, Proceedings of the National Academy of Sciences USA 80: 21-25). Other even stronger promoters for use in E. coli are the bacteriophage promoters from T7 and T5 phages. The T7 promoter requires the presence of the T7 polymerase in the E. coli host (Studier and Moffatt, J. Mol. Biol. 189, 113, (1986)). All these promoters are regulated by induction with IPTG, lactose or tryptophan to initiate transcription at strategic points in the bacterial growth period. E. coli also has strong promoters for continuous expression, eg. the synthetic promoter used to express hGH in Dalbøge et al, 1987, Biotechnology 5, 161-164.

For the expression in Bacillus, the promoters from Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes are suitable examples. Further promoters are described in “Useful proteins from recombinant bacteria” in Scientific American, 1980, 242: 74-94; and in Sambrook et al., 1989, supra.

Effective signal peptide coding regions for bacterial host cells are, for E. coli, the signal peptides obtained from the genes DegP, OmpA, OmpF, OmpT, PhoA and Enterotoxin STII, all from E. coli. For Bacillus the signal peptide regions obtained from Bacillus NCIB 11837 maltogenic amylase, Bacillus stearothermophilus alpha-amylase, Bacillus licheniformis subtilisin, Bacillus licheniformis beta-lactamase, Bacillus stearothermophilus neutral proteases (nprT, nprS, nprM) and Bacillus subtilis prsA. Further signal peptides are described by Simonen and Palva, 1993, Microbiological Reviews 57: 109-137. For both E. coli and Bacillus, signal peptides can be created de novo according to the rules outlined in the algorithm SignalP (Nielsen et al, 1997, Protein Eng. 10, 1-6., Emanuelsen et al, 2007, Nature Protocols 2, 953-971). The signal sequences are adapted to the given context and checked for SignalP score.

Examples of strong terminators for transcription are the aspartase aspA as in the Thiofusion Expression System, the T7 gene 10 terminator in the pET vectors (Studier et al) and the terminators of the ribosomal RNA genes rrnA, rrnD.

Examples of preferred expression hosts are E. coli K12 W3110, E. coli K12 with a trace of B, MC1061 and E. coli B BL21 DE3, harbouring the T7 polymerase by lysogenization with bacteriophage λ. These hosts are selectable with antibiotics when transformed with plasmids for expression. For antibiotics free selection the preferred host is e.g. E. coli B BL21 DE3 3xKO with deletion of the 2 D,L-alanine racemase genes Δalr, ΔdadX, and deletion of the Group II capsular gene cluster Δ (kpsM-kpsF), specific for E. coli B and often associated with pathogenic behaviour. The deletion of the Group II gene cluster brings E. coli B BL21 DE3 3xKO into the same safety category as E. coli K12. Selection is based on non-requirement of D-alanine provided by the alr gene inserted in the expression plasmid instead of the AmpR gene.

Once the bifunctional fusion protease has been expressed in a host organism it may be recovered and purified to the required purity by conventional techniques. Non-limiting examples of such conventional recovery and purification techniques are centrifugation, solubilization, filtration, precipitation, ion-exchange chromatography, immobilized metal affinity chromatography (IMAC), RP-HPLC, gel-filtration and freeze drying.

Examples of recombinant expression and purification of HRV14 3C may be found in e.g. Cordingley et al., J. Virol. 1989, 63, pp 5037-5045, Birch et al., Protein Expr Purif., 1995, 6, pp 609-618 and in WO2008/043847.

Examples of microbial expression and purification of XaaProDAP from Lactococcus lactis may be found in e.g. Chich et al, Anal. Biochem, 1995, 224, pp 245-249 and Xin et al., Protein Expr. Purif. 2002, 24, pp 530-538.

The invention is further described by the following non-limiting embodiments:
1. Bifunctional fusion enzyme comprising the catalytic domains of a picornaviral 3C protease and a XaaProDAP.
2. Bifunctional fusion protease according to embodiment 1, comprising a protein of the formula:

X—Y—Z (I) or

Z—Y—X (II)
wherein
X is a picornaviral 3C protease or a functional variant thereof;
Y is an optional linker;
Z is a Xaa-Pro-dipeptidyl aminopeptidase (XaaProDAP) or a functional variant thereof; wherein said fusion protease has substantially no self-cleavage activity able to deteriorate at least one of the two proteolytic activities.
3. The bifunctional fusion protease according to any of embodiments 1-2 having the formula (I), i.e. said picornaviral 3C protease or a functional variant thereof is in the N-terminal part of said bifunctional fusion protease.
4. The bifunctional fusion protease according to any of embodiments 1-3, wherein X is a rhinoviral protease or a functional variant thereof.
5. The bifunctional fusion protease according to any of embodiments 1-3, wherein X is a picornaviral protease or a functional variant thereof.
6. The bifunctional fusion protease according to any of embodiments 1-4, wherein X is HRV14 3C or a functional variant thereof.
7. The bifunctional fusion protease according to any of embodiments 1-6, wherein X comprises SEQ ID NO:2, or a functional variant thereof.
8. The bifunctional fusion protease according to any of embodiments 5-6, wherein X is P2X₁—SEQ ID NO:2, where X₁ is selected from the genetically encoded amino acid residues but P, or G1P—SEQ ID NO:2, or a functional variant thereof.
9. The bifunctional fusion protease according to any of embodiments 5-6, wherein X is CVB3 3C or a functional variant thereof.
10. The bifunctional fusion protease according to embodiment 5, wherein X comprises SEQ ID NO:23, or a functional variant thereof.
11. The bifunctional fusion protease according to any of embodiments 1-10, wherein X is a C-terminally truncated functional picornaviral 3C protease or a functional variant thereof.
12. The bifunctional fusion protease according to embodiment 11, wherein said C-terminally truncated functional picornaviral 3C protease has been truncated by no more than 20 amino acid residues, such as no more than 10 amino acid residues, such as no more than 5 amino acid residues, such as no more than 2 amino acid residues.
13. The bifunctional fusion protease according to any of embodiments 1-12, wherein X is an enzyme from a virus selected from Enterovirus, Coxsackievirus, Cowpea mosaic comovirus, Rhinovirus and Poliovirus, or a functional variant thereof.
14. The bifunctional fusion protease according to any of embodiments 1-13, wherein Z is an E.C. 3.4.14.11 enzyme or a functional variant thereof.
15. The bifunctional fusion protease according to embodiment 14, wherein Z is an enzyme from a lactic acid bacterium or a functional variant thereof.
16. The bifunctional fusion protease according to embodiment 15, wherein Z is an enzyme from Lactococcus spp., Streptococcus spp., Lactobacillus spp., Bifidobacterium spp. or a functional variant thereof.
17. The bifunctional fusion protease according to any of embodiments 1-16, wherein Z is SEQ ID NO:1 or a functional variant thereof.
18. The bifunctional fusion protease according to any of embodiments 1-14, wherein Z is an enzyme from a Bacillus spp., or a functional variant thereof.
19. The bifunctional fusion protease according to any of embodiments 1-16, wherein Z is an enzyme from Streptococcus suis, or a functional variant thereof.
20. The bifunctional fusion protease according to embodiment 17, wherein Z is SEQ ID NO: 24 or a functional variant thereof.
21. The bifunctional fusion protease according to any of embodiments 1-17, wherein Z is an enzyme from Lactococcus lactis, or a functional variant thereof.
22. The bifunctional fusion protease according to any of embodiments 1-13, wherein said Z is an E.C. 3.4.14.5 enzyme or a functional variant thereof.
23. The bifunctional fusion protease according to any of embodiments 1-22, wherein Z is a protein having an exposed loop connecting two alpha-helixes.
24. The bifunctional fusion protease according to embodiment 23, wherein said loop does not comprise any QG subsequence.
25. The bifunctional fusion protease according to any of embodiments 23-24, wherein said loop does not comprise any of the subsequences QS, QI, QN, QA and QT.
26. The bifunctional fusion protease according to any of embodiments 23-25, wherein said loop is the sequence spanning the amino acid residues 223 to 270 in SEQ ID NO:1.
27. The bifunctional fusion protease according to any of embodiments 23-26, wherein said loop is the sequence in a XaaProDAP which corresponds to the sequence spanning the amino acid residues 223 to 270 in SEQ ID NO:1.
28. The bifunctional fusion protease according to any of embodiments 23-27, wherein said loop is the sequence having at least 70% amino acid identity with the sequence spanning amino acid residues 223 to 270 in SEQ ID NO:1.
29. The bifunctional fusion protease according to any of embodiments 1-28, wherein Z comprises no more than one QG subsequence.
30. The bifunctional fusion protease according to any of embodiments 1-29, wherein Z does not comprise any QG subsequence.
31. The bifunctional fusion protease according to any of embodiments 1-17, wherein Z comprises at least one substitution, addition or deletion of an amino acid residue in Q241-G242.
32. The bifunctional fusion protease according to embodiment 31, wherein Z comprises the substitutions Q241E, G242T.
33. The bifunctional fusion protease according to any of embodiments 1-32, wherein the second amino acid residue from the N-terminal in said fusion protease is different from P.
34. The bifunctional fusion protease according to any of embodiments 1-33, wherein the second amino acid residue from the N-terminal in said fusion protease is different from G, A and T.
35. The bifunctional fusion protease according to any of embodiments 1-33, wherein the N-terminal in said fusion protease has the amino acid sequence MX₁P, where X₁ is an amino acid rendering the MX₁P sequence a poor substrate for methionine aminopeptidase.
36. The bifunctional fusion protease according to any one of embodiments 1-34, wherein the N-terminal amino acid residue in said bifunctional fusion protease is P.
37. The bifunctional fusion protease according to embodiment 36, wherein the second amino acid residue from the N-terminal in said fusion protease is not P, G, A or T.
38. The bifunctional fusion protease according to any of embodiments 1-37, which comprises no linker Y.
39. The bifunctional fusion protease according to any of embodiments 1-37, which comprises a linker Y.
40. The bifunctional fusion protease according to embodiment 39, wherein said linker Y has a length of from 2 to 100 amino acid residues.
41. The bifunctional fusion protease according to any of embodiments 39-40, wherein said linker Y has a length of from 2 to 50 amino acid residues.
42. The bifunctional fusion protease according to any of embodiments 39-41, wherein said linker Y has a length of from 2 to 25 amino acid residues.
43. The bifunctional fusion protease according to any of embodiments 39-42, wherein said linker Y has a length of from 2 to 15 amino acid residues.
44. The bifunctional fusion protease according to any of embodiments 39-41, wherein Y has a length of from about 5 to about 50 amino acid residues.
45. The bifunctional fusion protease according to any of embodiments 38-39, wherein Y has a length of from about 5 to about 15 amino acid residues.
46. The bifunctional fusion protease according to any of embodiments 39-45, wherein Y comprises no Cys residues.
47. The bifunctional fusion protease according to any of embodiments 39-46, wherein Y comprises no Gln residues.
48. The bifunctional fusion protease according to any of embodiments 39-47, wherein Y comprises only the following amino acid residues: G, S, A, L, P and T.
49. The bifunctional fusion protease according to any of embodiments 39-48, wherein Y is selected from the group consisting of SEQ ID NOs 3, 4 and 12.
50. The bifunctional fusion protease according to any of embodiments 1-49, which is formula (I), i.e. said picornaviral 3C protease or a functional variant thereof is in the N-terminal part of said bifunctional fusion protease.
51. The bifunctional fusion protease according to embodiment 50, wherein X does not have a C-terminal amino acid residue which is Q.
52. The bifunctional fusion protease according to any of embodiments 1-49, which is formula (II). i.e. said picornaviral 3C protease or a functional variant thereof is in the C-terminal part of said bifunctional fusion protease.
53. The bifunctional fusion protease according to any of embodiments 1-52, which comprises a tag protein attached to the N-terminal.
54. The bifunctional fusion protease according to embodiment 53, wherein said tag protein is selected from the group consisting of a His-tag, a solubilisation domain and a His-tagged solubilisation domain.
55. The bifunctional fusion protease according to any of embodiments 1-54, wherein said functional variant comprises from 1-2 amino acid substitutions, deletions or additions or from 1-5 amino acid substitutions, deletions or additions, or from 1-15 amino acid substitutions, deletions or additions relative to the corresponding naturally occurring protein or naturally occurring sub-sequence.
56. The bifunctional fusion protease according to any of embodiments 1-55, wherein the determination of said fusion protease having substantially no self-cleavage activity able to deteriorate at least one of the two proteolytic activities is determined by said bifunctional fusion protease being suitable for the intended use thereof.
57. The bifunctional fusion protease according to any of embodiments 1-55, wherein the determination of said fusion protease having substantially no self-cleavage activity able to deteriorate at least one of the two proteolytic activities is determined by at least 50% of the bifunctional fusion protease being intact after incubating said bifunctional fusion protease at a concentration of 0.5 mg/mL, in 1×PBS buffer, pH 7.4 at the temperature 37° C. for 3 hours.
58. The bifunctional fusion protease according to any of embodiments 1-55, wherein the determination of said fusion protease having substantially no self-cleavage activity able to deteriorate at least one of the two proteolytic activities is determined by at least 50% of both the picornaviral 3C protease activity and the XaaProDAP activity of the bifunctional fusion protease being intact after incubating said bifunctional fusion protease at a concentration of 0.5 mg/mL, in 1×PBS buffer, pH 7.4 at the temperature 37° C. for 3 hours.
59. The bifunctional fusion protease according to embodiment 58, wherein at least 80% of both the picornaviral 3C protease activity and the XaaProDAP activity of the bifunctional fusion protease being intact after incubating said bifunctional fusion protease at a concentration of 0.5 mg/mL, in 1×PBS buffer, pH 7.4 at the temperature 37° C. for 3 hours.
60. The bifunctional fusion protease according to any of embodiments 1-55, wherein the determination of said fusion protease having substantially no self-cleavage activity able to deteriorate at least one of the two proteolytic activities is determined by at least 50% of both the picornaviral 3C protease activity and the XaaProDAP activity of the bifunctional fusion protease being intact after incubating said bifunctional fusion protease at a concentration of 0.5 mg/mL, in 1×PBS buffer, pH 7.4 at the temperature 4° C. for 24 hours.
61. The bifunctional fusion protease according to embodiment 60, wherein at least 80% of both the picornaviral 3C protease activity and the XaaProDAP activity of the bifunctional fusion protease being intact after incubating said bifunctional fusion protease at a concentration of 0.5 mg/mL, in 1×PBS buffer, pH 7.4 at the temperature 4° C. for 24 hours.
62. Method for preparing a bifunctional fusion protease according to any of embodiments 1-61, comprising the recombinant expression of a protein comprising the bifunctional fusion protease in a host cell and subsequently isolating the bifunctional fusion protease.
63. The method according to embodiment 62 wherein said host cell is E. coli.
64. The method according to any of embodiments 62-63 wherein said bifunctional fusion protease is isolated as a soluble protein.
65. The method according to any of embodiments 62-64 wherein said bifunctional fusion protease is isolated as a soluble protein without the use of a refolding step.
66. The method according to any of embodiments 62-65 wherein said bifunctional fusion protease has the formula (I) as depicted in embodiment 2, i.e. said picornaviral 3C protease or a functional variant thereof is in the N-terminal part of said bifunctional fusion protease.
67. Use of the bifunctional fusion protease according to any of embodiments 1-66 for removing an N-terminal peptide or protein from a larger peptide or protein.
68. The use according to embodiment 67, wherein said larger peptide or protein is contacted with said bifunctional fusion protease under suitable reaction conditions and for sufficient time to liberate the majority of said N-terminal peptide.
69. The use according to any of embodiments 67-68 wherein the bifunctional fusion protease is co-expressed with said larger peptide or protein to release the protein of interest in vivo during expression in a host cell.
70. The use according to any of embodiments 67-68 wherein said larger peptide or protein is contacted with said bifunctional fusion protease following isolation of these two proteins from the host cells used for their expression.
71. The use according to any of embodiments 67-70 wherein said larger peptide or protein is selected from peptides or proteins comprising a peptide selected from GLP-1, glucagon, PYY, amylin and functional variants thereof.
72. The use according to any of embodiments 67-71 wherein said larger peptide or protein has a size of less than 200 amino acid residues, less than 150 amino acid residues, less than 100 residues, or less than 60 amino acid residues.

### EXAMPLES

### Example 1

### Plasmid Constructs and Expression of HRV14/XaaProDAP or XaaProDAP/HRV14 Variants

The pET system was used for expression of enzymes as this system provides a powerful approach for expressing proteins in E. coli. In pET vectors, target genes are cloned under control of strong bacteriophage T7 transcription and translation signals, and expression is induced by providing a source of T7 RNA polymerase in the host cell.

E. coli expression plasmids (pET22b, Novagen) encoding bifunctional fusion proteases comprising fusions of the HRV14 3C and the Lactococcus lactis XaaProDAP sequence. In one set of constructs the HRV14 3C part was positioned in the N-terminal of XaaProDAP sequence using an intervening linker GGSGGSGGS (SEQ ID NO: 3) to separate the two domains (Table 1).

TABLE 1
pET22b plasmid constructs encoding NH2-HRV14 3C-XaaProDAP-
COOH fusion proteases.

| | | | HRV14 | | | |
|---|---|---|---|---|---|---|
| | | | 3C | Gly- | XaaProDAP | |
| | Product | Fusion | domain | Ser | enzyme | GSS |
| Protease | name | partner | (N-term) | linker | (C-term) | extension |
| 12756 | His- | SEQ | SEQ ID | SEQ | SEQ ID | GSS |
| | HRV14- | ID | NO: 2 | ID | NO: 1 | |
| | XaaProDAP | NO: 5 | | NO: 3 | | |
| 12757 | DsbC- | SEQ | SEQ ID | SEQ | SEQ ID | GSS |
| | HRV14- | ID | NO: 2 | ID | NO: 1 | |
| | XaaProDAP | NO: 6 | | NO: 3 | | |
| 12758 | RL9- | SEQ | SEQ ID | SEQ | SEQ ID | GSS |
| | HRV14- | ID | NO: 2 | ID | NO: 1 | |
| | XaaProDAP | NO: 7 | | NO: 3 | | |
| 12759 | NusA- | SEQ | SEQ ID | SEQ | SEQ ID | GSS |
| | HRV14- | ID | NO: 2 | ID | NO: 1 | |
| | XaaProDAP | NO: 8 | | NO: 3 | | |
| 12760 | His-MBP2- | SEQ | SEQ ID | SEQ | SEQ ID | GSS |
| | HRV14- | ID | NO: 2 | ID | NO: 1 | |
| | XaaProDAP | NO: 9 | | NO: 3 | | |
| 12761 | His-Trx- | SEQ | SEQ ID | SEQ | SEQ ID | GSS |
| | HRV14- | ID | NO: 2 | ID | NO: 1 | |
| | XaaProDAP | NO: 10 | | NO: 3 | | |

In another set of plasmids encoding fusion proteases, the HRV14 3C part was placed in the C-terminal of the XaaProDAP sequence with an intervening linker GSSGSGGSG (SEQ ID NO: 4) separating the two domains.

TABLE 2
pET22b plasmid constructs encoding NH2-XaaProDAP-HRV14 3C-
COOH fusion proteases.

| | | | | | | HRV14 |
|---|---|---|---|---|---|---|
| | | | Gly- | XaaProDAP | | 3C |
| | Product | Fusion | Ser | enzyme | GS | domain |
| Protease | name | partner | linker | (C-term) | linker | (N-term) |
| 12768 | His- | SEQ ID | GS | SEQ ID | SEQ ID | SEQ |
| | XaaProDAP- | NO: 5 | | NO: 1 | NO: 4 | ID |
| | HRV14 | | | | | NO: 2 |
| 12769 | DsbC-His- | SEQ ID | GS | SEQ ID | SEQ ID | SEQ |
| | XaaProDAP- | NO: 6 | | NO: 1 | NO: 4 | ID |
| | HRV14, 3C | | | | | NO: 2 |
| 12770 | RL9-His- | SEQ ID: | GS | SEQ ID | SEQ ID | SEQ |
| | XaaProDAP- | NO: 7 | | NO: 1 | NO: 4 | ID |
| | HRV14 | | | | | NO: 2 |
| 12771 | NusA-His- | SEQ ID | GS | SEQ ID | SEQ ID | SEQ |
| | XaaProDAP- | NO: 8 | | NO: 1 | NO: 4 | ID |
| | HRV14 | | | | | NO: 2 |
| 12772 | MBP2- | SEQ ID | GS | SEQ ID | SEQ ID | SEQ |
| | XaaProDAP- | NO: 9 | | NO: 1 | NO: 4 | ID |
| | HRV14 | | | | | NO: 2 |
| 12773 | His-Trx- | SEQ ID | GS | SEQ ID | SEQ ID | SEQ |
| | XaaProDAP- | NO: 10 | | NO: 1 | NO: 4 | ID |
| | HRV14 | | | | | NO: 2 |

Expression, purification or solubility enhancing fusion partners were placed in the N-terminal of both variants of the bifunctional protease. The fusions partners were designed to comprise a His6 tag (either in the N- or C-terminal of the fusion partner sequence) and a sequence encoding a flexible Gly-Ser-rich linker, comprising a Hepatitis A Virus 3C protease (HAV) cleavage site with the sequence GGSSGSGSELRTQS (SEQ ID NO: 22) introduced adjacent to the N-terminal amino acid of the bifunctional protease sequence, to allow enzymatic separation of the fusion partner from the protease part if needed.

The gene fragments encoding the fusion proteases described in Table 1 and 2 were codon-optimized for expression in E. coli and prepared by gene synthesis (GenScript). The plasmid constructs specified in Table 1 and 2 were generated by inserting the synthetic gene fragments into pET22b vectors using standard cloning technologies known to those of ordinary skill in the art (obtained from GenScript)

### Evaluation of the Fusion Protease Variants by Small Scale Expression and Purification

Expression plasmids were transformed into E. coli BL21(DE3) (Novagen) and expressed in small scale.

E. coli BL21(DE3) were transformed with plasmid using a procedure based on Heat Shock at 42° C. according to the manufacturer. Transformed cells were plated onto LB agar plates and incubate overnight at 37° C. with 10 mg/L ampicillin. Overnight Terrific broth (TB) culture with 0.5% glucose and 50 mg/L carbenicillin of each transformant was prepared at 30° C. and shaking at 700 rpm using a Glas-Col shaker (Glas-Col). 20 μL of overnight culture of each transformant was used to inoculate 0.95 μL of TB medium with 50 mg/L carbenicillin in 96 Deep-Well plates (2 ml) and transformants were propagated overnight at 700 rpm. Expression cultures were incubated at 37° C. until OD600 of 1.5 was reached. The cultures were then cooled to 20° C. and protein induction was carried out overnight using 0.3 mM IPTG. Pellets containing expressed protein were harvested by centrifugation at 1800×G.

Purification screen: Small scale purification using IMAC resin was performed to evaluate the combined expression and purification potential and the integrity of the proteases. In short, 250 μL of lysis buffer (50 mM NaPO4, 300 mM NaCl, 10 mM Imidiazole, 10 mg/ml Lysozyme, 250 U/μL Benzoase and 10% DDM (dodecyl matoside)) was added to each pellet and the cells were lyzed using freeze/thaw cycles. Debris was removed by centrifugation, the supernatant was filtered (0.45 μm) and transferred onto 1.2 μm filter plates containing Ni2+-loaded Sepharose Fast Flow (prepared from washing 30 μL of a 50% slurry in 20% EtOH) (GE Healthcare). The supernatant was incubated for 20 min by shaking at 400 rpm with resin to bind the protein and the solute was removed by gentle centrifugation at 100×g for 1 min. The resin was washed with 50 mM sodium phosphate, 300 mM NaCl, 30 mM Imidiazole, pH 7.5 by gentle mixing and the resin was dried by centrifugation. To elute the protein, 40 μL of elution buffer (50 mM sodium phosphate, 300 mM NaCl, 300 mM Imidazole) was added to the resin, incubated for 10 min by shaking at 400 rpm and the eluate containing partly purified enzymes was collected.

Whole lysates of pellets from the expression of fusion protease variants were analysed by SDS-PAGE. For none of the fusion protease variants described in Table 1 or 2, significant amounts of full-length protein could be observed. For several of the fusion protease variants, clear bands of different sizes were however observed. SDS-PAGE analysis of IMAC purified samples was consistent with these observation as it did also not indicate production of a full length proteases, but rather bands of smaller sizes. The observations indicates that the fusion proteases are truncated or degraded during expression and/or following capture on IMAC resin. As distinct bands were observed for several fusion protease variants and the expression level appeared to be significant based on gel band intensities, the absence of full-length protein is rather due to unintended hydrolysis at specific positions in the fusion proteases resulting in a significant truncation of the fusion proteases.

### LC-MS Analysis of Fusion Proteases

Eventual cleavage sites, which could explain the truncated forms of fusion proteases, observed occurring was detected by mass spectrometry using a MaXis Impact Ultra high resolution time-of-flight (UHR-TOF) mass spectrometer (Bruker Daltonics) equipped with a Dionex UltiMate3000™ liquid chromatometer (Dionex) allowing Diode array measurements at UV215 nm with general settings according to the instructions of the manufacturer.
Enzymes were separated on a Waters Aquity BEH300 C4 Reversed phase 1.0×100 mm column with 1.7 μm pore size using a column temperature of 45° C. and a flow rate of 0.2 ml/min. The solvents used were are follows
Solvent A: 0.1% formic acid in H₂O
Solvent B: 99.9% MeCN, 0.1% formic acid (v/v)
Liquid Chromatography was performed with the following gradient to separate the enzyme digests.

| Time (min) | % A | % B |
|---|---|---|
| 0 | 90 | 10 |
| 2 | 90 | 10 |
| 10 | 10 | 90 |
| 11 | 10 | 90 |
| 12 | 90 | 10 |
| 13 | 90 | 10 |
| 14 | 50 | 50 |

The recorded mass spectra were deconvoluted and analysed using the Bruker Compass data analysis version 4.1 software (Bruker Daltonics) covering mass ranges from 10.000 Da to 140.000 Da and resolutions (>10.000) according to manufacturer instructions. The UV215 nm chromatogram and total ion count (TIC) chromatograms were evaluated in parallel, to ensure that there was agreement between MS data obtained and UV215 nm traces of the peptides. The experimental determined masses indicated refers to the average isotopic mass and the mass spectrometry data was obtained with a mass accuracy better than 200 ppm.

When Protease 12756 was analysed a mass of 22241.54 Da was detected. This mass corresponds to the mass of the His6 fusion partner (SEQ ID NO: 5) and the HRV14 3C domain (SEQ ID NO:2) (calculated mass 22242.27 Da). Thus, a cleavage site occurred between Gln/Gly in the junction of the C-terminal of the HRV14 3C domain (SEQ ID NO:2) and the N-terminal of the linker (SEQ ID NO: 3). This indicated that 3C protease was able to excise itself out of the fusion protease in which HRV14 3C was fused to the N-terminal of XaaProDAP, in a similar way as it has been reported to do from its natural viral polyprotein. This was also observed for fusion proteases 12757, 12758, 12760 and 12761 with size variations corresponding to differences in the size of the N-terminal fusion partner used.

### Example 2

### Removal of C-Terminal Q182 in HRV14 Domain of NH2-HRV14-XaaProDAP-COOH Fusion Proteases

For fusion protease variants shown in Table 1, it was observed that fragments often corresponded in size to the fusion partner plus the HRV14 3C domain sequence. To remove this possibility for cleavage, a new linker was designed to replace the original GS linker (SEQ ID NO:3) between HRV14 3C and XaaProDAP domains in the fusion proteases comprising His6, RL9 or Trx fusion partners (Table 1, Example 1). The Gln/Gly cleavage site in the junction of the HRV14 enzyme and start of SEQ ID NO:3 was replaced by Ser-Gly. Thus, the last amino acid (Gln182) in the HRV14 3C protease domain was removed to yield des182-HRV14 3C with the following sequence: GPNTEFALSLLRKNIMTITTSKGEFTGLGIHDRVCVIPTHAQPGDDVLVNGQKIRVKDKYKLV DPENINLELTVLTLDRNEKFRDIRGFISEDLEGVDATLVVHSNNFTNTILEVGPVTMAGLINLS STPTNRMIRYDYATKTGQCGGVLCATGKIFGIHVGGNGRQGFSAQLKKQYFVEK (SEQ ID NO: 11) and the Gly in the beginning of the linker (SEQ ID NO:3) was removed as this site represents a cleavage site for the 3C protease. Instead the linker between the HRV14 domain (SEQ ID NO:11) and the XaaProDAP domain was replaced with SGSGGSGGSGS (SEQ ID NO:12). The new fusion protease variants are depicted in Table 3:

TABLE 3
pET22b plasmid constructs encoding des182HRV14 3C-XaaProDAP
fusion proteases.

| | | | HRV14 3C | | XaaProDAP | |
|---|---|---|---|---|---|---|
| | Product | Fusion | domain | Gly-Ser | enzyme | GSS |
| Protease | name | partner | (N-term) | linker | (C-term) | extension |
| 20177 | His-des182HRV14 | SEQ ID | SEQ ID | SEQ ID | SEQ ID | GSS |
| | XaaProDAP | NO: 5 | NO: 11 | NO: 12 | NO: 1 | |
| 20397 | RL9-des182HRV1 | SEQ ID: | SEQ ID | SEQ ID | SEQ ID | GSS |
| | 4XaaProDAP | NO: 7 | NO: 11 | NO: 12 | NO: 1 | |
| 20400 | His-Trx-des182HRV1 | SEQ ID | SEQ ID | SEQ ID | SEQ ID | GSS |
| | 4XaaProDAP | NO: 10 | NO: 11 | NO: 12 | NO: 1 | |

Small scale expression and purification of these constructs were done as described in Example 1. SDS-PAGE of samples from IMAC purification, showed that two clearly visible and predominant bands around 50-60 kDa now occurred for these three fusion protease variants indicating that the full-length protease was cleaved into two fragments. LC-MS analysis was performed as described in Example 1 to pinpoint the cleavage site. Analysis of Protease 20177 showed that this fusion protease variant was cleaved into two major bands which had a mass of 51091.27 Da and 59773.49 Da. These masses demonstrated that another cleavage site appeared between Gln241 and Gly242 in the XaaProDAP sequence (SEQ ID NO:1) as the determined masses were in agreement with the calculated masses for these fragments, as deducted from the Protease 20177 amino acid sequence (51092.15 Da and 59772.43 Da, respectively). The exact same cleavage site was clearly observed by analysis of deconvoluted spectra for all three constructs depicted in Table 3, thus indicating that this site was highly sensitive regardless of which N-terminal fusion partner was used. Upon evaluation of the available 3D structure (Rigolet et al., Structure, 10, pp 1384-1394) it could be determined that the cleavage site occurred in the middle of a very large loop connecting two small alpha-helixes in the catalytical domain of L. lactis XaaProDAP spanning from approximately aa residue 223-270. This loop is highly exposed and therefore sensitive for cleavage and the Q/G sequence indicates that the 3C protease itself is responsible for this cleavage. Another less predominant unwanted cleavage site was observed in the Gln/Ser position in cleavage site for the HAV protease (ELRTQ/S) in the C-terminal of the fusion partners could also be detected by analysis of the IMAC purified samples

### Example 3A

### Design of Full-Length Bifunctional NH2-HRV14-XaaProDAP-COOH Proteases

In order to remove the cleavage site observed between Gln241 and Gly242 in the XaaProDAP sequence in Example 2, the two amino acids were substituted with Glu241 and Thr242. The Glu241-Thr242 substitution was chosen as replacement for Gln241-Gly242 as it occurred as a natural aa variation on basis of homology searches of orthologs of XaaProDAP from different isolates of L. lactis. As undesired cleavage also occurred in the HAV site in the C-terminal of fusion partners, the HAV site was replaced with a small GS containing sequence. These fusion partners had the sequence MHHHHHHGGSSGSGSGSGSGS (SEQ ID NO: 13), MKVILLRDVPKIGKKGEIKEVSDGYARNYLIPRGFAKEYTEGLERAIKHEKEIEKRKKEREREE SEKILKELKKRTHVVKVKAGEGGKIFGAVTAATVAEEISKTTGLKLDKRWFKLDKPIKELGEY SLEVSLPGGVKDTIKIRVEREEGSGSGHHHHHHGGSSGSGSGSGSGS (SEQ ID NO:14) and MHHHHHHGSGSGSDKIIHLTDDSFDTDVLKADGAILVDFWAEWCGPCKMIAPILDEIADEYQ GKLTVAKLNIDQNPGTAPKYGIRGIPTLLLFKNGEVAATKVGALSKGQLKEFLDANLAGGSSG SGSGSGSGS (SEQ ID NO: 15)

Plasmid constructs comprising the Q241E, G242T substitution and removal of the the HAV site from the linker in front of the HRV14 3C domain were obtained (Genscript). The constructs designed and tested are depicted in Table 4.

Small scale expression and IMAC purification of the new fusion protease constructs were conducted as described in Example 1. From SDS-PAGE analysis it was observed, that the Q241E and G242T substitution, clearly prevented the cleavage of the fusion protease into the two parts. Very intense gel bands of approximately 100-120 kDa was observed for all three constructs, showing that the Q241E,G242T substitution resulted in production of soluble and intact full-length fusion proteases comprising both the HRV14 3C domain and the XaaProDAP domain. The benefit of removing the ELRTQ site (by substitution with GSGSG) was less pronounced in this experiment.

LC-MS of the fusion protease variants in Table 4 was conducted as described in Example 1 and confirmed the observations from SDS-PAGE. Protease 20986, 20988 and 20990 had determined masses of 110604.97 Da, 127867.76 Da and 122607.21 Da, respectively, which are in agreement with the calculated masses 110605.18 Da, 127867.23 Da and 122605.91 Da, respectively. Thus, the modified fusion proteases in Table 4 were not significantly truncated or degraded, as the predominant detected masses corresponding to the calculated mass for the full-length fusion proteases.

TABLE 4
pET22b plasmid constructs encoding NH2-des182HRV14 3C-XaaProDAP
(Q241E, G242T)-COOH fusion enzymes.

| | | | HRV14 3C | | XaaProDAP | |
|---|---|---|---|---|---|---|
| | | Fusion | domain | Gly-Ser | enzyme | GSS |
| Protease | Product name | partner | (N-term) | linker | (C-term) | extension |
| 20986 | His- | SEQ ID | SEQ ID | SEQ ID | SEQ ID | GSS |
| | des182HRV14- | NO: 13 | NO: 11 | NO: 12 | NO: 1 | |
| | XaaProDAP | | | | (Q241E, | |
| | (Q241E, G242T) | | | | G242T) | |
| 20988 | RL9- | SEQ ID: | SEQ ID | SEQ ID | SEQ ID | GSS |
| | des182HRV14- | NO: 14 | NO: 11 | NO: 12 | NO: 1 | |
| | XaaProDAP | | | | (Q241E, | |
| | (Q241E, G242T) | | | | G242T) | |
| 20990 | His-Trx- | SEQ ID | SEQ ID | SEQ ID | SEQ ID | GSS |
| | des182HRV14- | NO: 15 | NO: 11 | NO: 12 | NO: 1 | |
| | XaaProDAP | | | | (Q241E, | |
| | (Q241E, G242T) | | | | G242T) | |

### Example 3B

### Design of Full-Length Bifunctional NH2-XaaProDAP-HRV14-COOH Proteases

Using the general design, cloning and expression procedures described in Example 1 and 3A we also evaluated whether a functional and soluble fusion protease comprising HRV14 3C in the C-terminal could be obtained. Expression of 3 fusion proteases were evaluated, which comprised a C-terminal HRV14 3C domain and an N-terminal XaaProDAP (Q241E,G242T) using previously described 3 different N-terminal tags (His6, RL9, Trx). All 3 constructs in which the HRV14 3C domain is placed in the C-terminal were expressed as insoluble protein as determined by SDS-PAGE of uninduced, induced, soluble and insoluble fractions (detailed data not shown). This demonstrate, that fusion protease variants comprising the HRV14 3C protease in the N-terminal and L. lactis XaaProDAP in the C-terminal surprisingly has a more optimal folding kinetics, which leads to a soluble and stable fusion protease, which is easier to produce and which does not require any cost prohibitive refolding steps. In conclusion, certain specifications of protein design made it possible to produce intact fusion proteases comprising a HRV14 3C and XaaProDAP protease.

### Example 4

### Scaling Up Expression and Purification of NH2-His-des182HRV14-LLXaaProDAP (Q241E,G242T)-COOH (Protein 20986)

In order to prepare a larger amount of full-length fusion protease, Protease 20986 was scaled up for further testing of activity.

BL21(DE3) transformants (from a glycerol stock) harbouring the pET22b plasmid encoding Protease 20986 was propagated overnight in 50 ml of Terrific Broth medium containing 50 mg/L Carbenicillin and 0.5% glucose by shaking at 37° C. with 100 rpm (Multitron Standard shaker, 50 mm amplitude, Infors HT). The following day, 7.5 ml overnight culture was used to inoculate 750 ml of TB medium with 50 mg/L Carbenicillin in a 2 L shaker flask and the culture was subsequently incubated at 37° C. with 100 rpm. When OD600 of ˜1.5 was reached, the culture was cooled to 20° C. for 30 min., before 0.3 mM IPTG was added to induce the protein. The induction was carried out overnight at 20° C. at 100 rpm, and cells were harvested by centrifugation at 4000×g for 10 minutes. Pelleted cells were frozen until usage.

### Purification of His-des182HRV14-LLXaaProDAP (Q241E,G242T) (Protein 20986)

In order to obtain purified bifunctional fusion protease for further analysis, two consecutive purification steps were conducted in order to purify Protease 20986

14.7 g of cell pellets were suspended in 100 ml lysis buffer containing 50 mM sodium phosphate pH 7.5 and 3 μL benzonase. The cells were disrupted in a cell homogenizer at 1.4 kBar for one cycle and cell debris was spun down at 18.000 g for 20 min. The supernatant was then sterile filtered (0.45 micrometer). The purification of Protease 20986 was done using an AKTAExpress (GE Healthcare) for two consecutive purification steps. In the capture step, enzyme from the 100 ml of sample application was purified on a 2×1 ml HisTrap crude column (GE Healthcare) with a flow rate of 0.8 ml/min using the following buffers:

Buffer A: 50 mM sodium phosphate, 300 mM NaCl, 10 mM imidazole pH 7.5

Buffer B: 50 mM sodium phosphate, 300 mM NaCl, 300 mM imidazole pH 7.5

Buffer C: 50 mM sodium phosphate, 300 mM NaCl, 30 mM imidazole pH 7.5

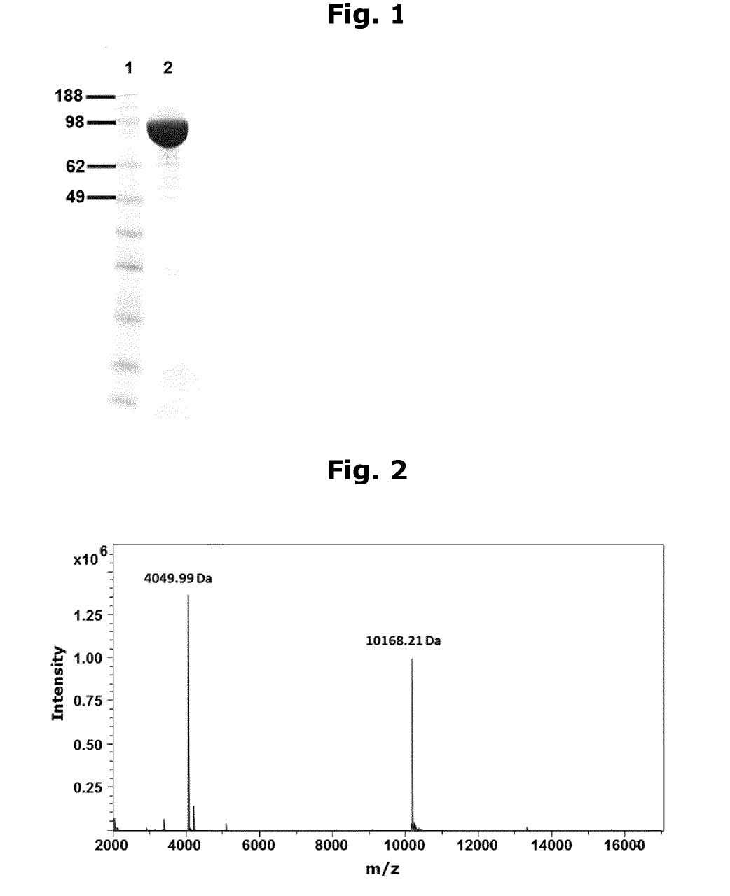

The column was initially equilibrated for 10 column volumes of buffer. After loading of the application, unbound protein was removed by washing using 7 column volumes of buffer C. A step elution from 0-100% buffer B for 5 column volumes was used to elute Protease 20986 the collected peak was stored in a loop and loaded onto a 120 ml HiLoad S200 16/600 (GE-Healthcare) gel filtration column. Size separation was performed with a flow rate of 1.2 ml/min using 1×PBS buffer (phosphate buffered saline, pH 7.4 with the composition 8.05 mM Na2HPO4x2H2O, 1.96 mM KH2PO4, 140 mM NaCl, pH 7.4).). Collected fraction of the predominant peak were analysed by SDS-PAGE and a clear band of the expected size around 100 kDa was observed. Fractions containing the highest amount of protease were pooled and the concentration was measured to be 1.6 mg/ml using UV280 measurements (NanoDrop, ThermoScientific). The purity was estimated to be higher than 90% as judged by SDS-PAGE (FIG. 1) and HPLC analysis.

### Example 5

### Plasmid Constructs and Expression of Model Fusion Proteins Containing Basic Tag

In order to test whether the bifunctional fusion protease could be used for removal of N-terminal tags, three different model fusion proteins were prepared to be used as protein substrates. A basic tag comprising Ribosomal Protein L27 from T. maritima, previously described in WO2008/043847 was used as a fusion partner and has the sequence MAHKKSGGVAKNGRDSLPKYLGVKVGDGQIVKAGNILVRQRGTRFYPGKNVGMGRDFTLF ALKDGRVKFETKNNKKYVSVYEE (SEQ ID NO: 16). The fusion proteins were designed so that the RL27 fusion partner can be removed by HRV14 3C enzyme and the remaining GP sequence can be removed by XaaProDAP.

A flexible linker containing a HRV14 cleavage site was used to link the basic tag to the model peptide sequences and had the sequence SSSGGSEVLFQGP (SEQ ID NO: 17). The model peptide sequences used were human Peptide YY 3-36 (PYY(3-36)), Glucagon and Glucagon-like peptide 1 (7-37,K34R) (GLP-1(7-37,K34R)) having the following sequences:

PYY(3-36):
(SEQ ID NO: 18)
IKPEAPGEDASPEELNRYYASLRHYLNLVTRQRY
Glucagon:
(SEQ ID NO: 19)
HSQGTFTSDYSKYLDSRRAQDFVQWLMNT
GLP-1(7-37, K34R):
(SEQ ID NO: 20)
HAEGTFTSDVSSYLEGQAAKEFIAWLVRGRG

E. coli expression plasmids (pET22b, Novagen) were prepared such that they encoded the three fusion proteins as specified in Table 5.

TABLE 5
Model fusion proteins encoded by plasmid
constructs using pET22b vectors.

| | Calculated | | | |
|---|---|---|---|---|
| | Molecular mass | | HRV14 | |
| Product name | (without Met) | RL27 | linker | Peptide |
| RL27_EVLFQGP_{—} | 14354.5 Da | SEQ ID | SEQ ID | SEQ ID |
| PYY(3-36) | | NO: 16 | NO: 17 | NO: 18 |
| RL27_EVLFQGP_{—} | 13787.1 Da | SEQ ID | SEQ ID | SEQ ID |
| Glucagon | | NO: 16 | NO: 17 | NO: 19 |
| RL27_EVLFQGP_{—} | 13688.1 Da | SEQ ID: | SEQ ID | SEQ ID |
| GLP1(7-37, K34R) | | NO: 16 | NO: 17 | NO: 20 |

Gene fragments codon-optimized for E. coli and spanning the entire fusion proteins were made by gene synthesis and ligated into the cloning site of the pET22b vector using standard cloning techniques (obtained from GenScript)

### Expression of Model Fusion Proteins

Expression of RL27_EVLFQGP_PYY(3-36) was done essentially as described for Protease 20986 in Example 4. In short, expression of RL27_EVLFQGP_Glucagon and RL27_EVLFQGP_GLP-1(7-37,K34R) was done as follows: E. coli BL21(DE3) was transformed with the plasmid and plated on LB agar plates containing 100 mg/L ampicillin and overnight cultures were dissolved in 10 ml LB medium and used to inoculate 750 ml LB with 50 mg/ml Carbenicillin in shaker flasks. Shaker flasks were incubated at 100 rpm at 37° C. When OD600 of 0.4 was reached protein expression was induced by adding 0.3 mM IPTG and cells were harvested by centrifugation following 3 hours incubation at 37° C.

### Purification of Model Fusion Proteins

In short, capture of the fusion proteins from supernatants resulting from cell disruption was done by cation exchange chromatography essentially as previously described (WO2008/043847) using a SP FF HiTrap 5 ml (GE Healthcare) column on an AKTA Express at a flow rate of 4 ml/min and the following buffers:

Buffer A: 50 mM sodium phosphate, pH 7.0
Buffer B: 50 mM sodium phosphate, 1000 mM NaCl, pH 7.0

In short, after sample loading, and a washing step, the fusion proteins were eluted from the columns using Buffer B. To increase purity of following capture, the proteins were purified by gel filtration essentially as described in Example 4, but using a S75 16/600 column (GE-Healthcare) for the separation. The purified proteins were evaluated by SDS-PAGE analysis and the correct intact mass was verified by LC-MS. UV280 was used to determine the concentration of the fusion proteins.

### Example 6

### Enzymatic Reaction with Protease 20986 and RL27_EVLFQGP_PYY(3-36) as Model Protein Substrate

The concentration RL27-HRV14-PYY(3-36) was adjusted to a concentration of 0.5 mg/ml in 1×PBS, pH 7.4. Enzymatic reaction were setup in reaction volumes of 22 μl using PBS, pH 7.4 as enzyme reaction buffer. Incubations of Protease 20986 with RL27-EVLFQGP-PYY(3-36) substrate was setup using molar enzyme to substrate ratios of 1:20 or 1:40, respectively, and the reactions were carried out for 3 hours at 37° C. (as depicted in Table 6). A purified variant of the HRV14 3C protease with an N-terminal tag (ribosomal L9 from T. maritima), described in WO2008/043847, was also included in the experiment. This protease, named RL9-HRV14 3C was used in the same molar ratio as Protease 20986, but only possesses HRV14 3C activity. RL9-HRV14 3C has the following sequence: MKVILLRDVPKIGKKGEIKEVSDGYARNYLIPRGFAKEYTEGLERAIKHEKEIEKRKKEREREE SEKILKELKKRTHVVKVKAGEGGKIFGAVTAATVAEEISKTTGLKLDKRWFKLDKPIKELGEY SLEVSLPGGVKDTIKIRVEREESSSGSSGSSGSSGPNTEFALSLLRKNIMTITTSKGEFTGLGI HDRVCVIPTHAQPGDDVLVNGQKIRVKDKYKLVDPENINLELTVLTLDRNEKFRDIRGFISED LEGVDATLVVHSNNFTNTILEVGPVTMAGLINLSSTPTNRMIRYDYATKTGQCGGVLCATGKI FGIHVGGNGRQGFSAQLKKQYFVEKQ (SEQ ID NO: 21). As a negative control the RL27-HRV14-PYY(3-36) substrate was also incubated in reaction buffer without protease. The enzymatic reactions were stopped by addition >0.5 M AcOH prior to LC-MS analysis.

Results with Protease 20986 Using RL27_HRV14_PYY(3-36) as Fusion Protein Model Substrate.

LC-MS analysis of enzymatic reactions was done essentially as described in Example 1 only that a C18 Aquity BEH300 C4 Reversed phase 1.0×100 mm column with 1.7 μm pore size was used to ensure sufficient separation and resolution of smaller peptides evaluated. The instrument was adjusted settings for mass ranges (2000-17000 Da) and resolutions (>20.000) according to manufacturers instructions. The UV215 nm chromatogram and total ion count (TIC) chromatograms were evaluated in parallel, to ensure that there was agreement between MS data obtained and UV215 nm traces of the peptides. The experimental determined masses indicated in the following examples refers to the most abundant mass, e.g. the mass of the molecule with the most highly represented isotope distribution, based on the natural abundance of the isotopes of the protein detected. In the following, the mass spectrometry data was obtained with a mass accuracy lower than 100 ppm.

Analysis of deconvoluted mass spectra showed that the RL27_EVLFQGP_PYY(3-36) fusion protein (control without enzyme) had a mass of 14354.17 Da. This was in agreement with the calculated mass (14354.5 Da) for the fusion protein without the Initiator Methionine.

The results of the different reactions are depicted in Table 6.

TABLE 6
Enzymatic reactions using Protease 20986 from Example 4 and
RL27_EVLFQGP_PYY(3-36) as substrate, all incubated for 3 hours
at 37° C. Experimentally determined predominant peaks detected in
deconvoluted mass spectra of reaction 1-4 are indicated.

| | | | | Determined | | |
|---|---|---|---|---|---|---|
| | | | | molecular | Calculated | |
| Reaction | | Molar | Predominant | masses | mass | |
| number | Enzyme | ratio | detected peaks | (Dalton) | (Dalton) | Corresponds to |
| Reaction 1 | Protease | 1:20 | Peak #1 | 4049.98 | 4050.1 | PYY3-36 |
| | 20986 | | | | | (SEQ ID NO: 18) |
| | | | Peak #2 | 10168.21 | 10168.4 | RL27 tag |
| Reaction 2 | Protease | 1:40 | Peak #1 | 4050.06 | 4050.1 | PYY(3-36) |
| | 20986 | | | | | (SEQ ID NO: 18) |
| | | | Peak#2 | 10168.20 | 10168.4 | RL27 tag |
| | | | Peak#3 | 4204 | 4204.1 | GP-PYY(3-36) |
| Reaction 3 | RL9- | 1:20 | Peak #1 | 4204.05 | 4204.1 | GP-PYY(3-36) |
| | HRV14 3C | | | | | |
| | | | Peak #2 | 10168.19 | 10168.4 | RL27 tag |
| Reaction 4 | RL9- | 1:40 | Peak #1 | 4204.08 | 4204.1 | GP-PYY(3-36) |
| | HRV14 3C | | | | | |
| | | | Peak #2 | 10168.23 | 10168.4 | RL27 tag |

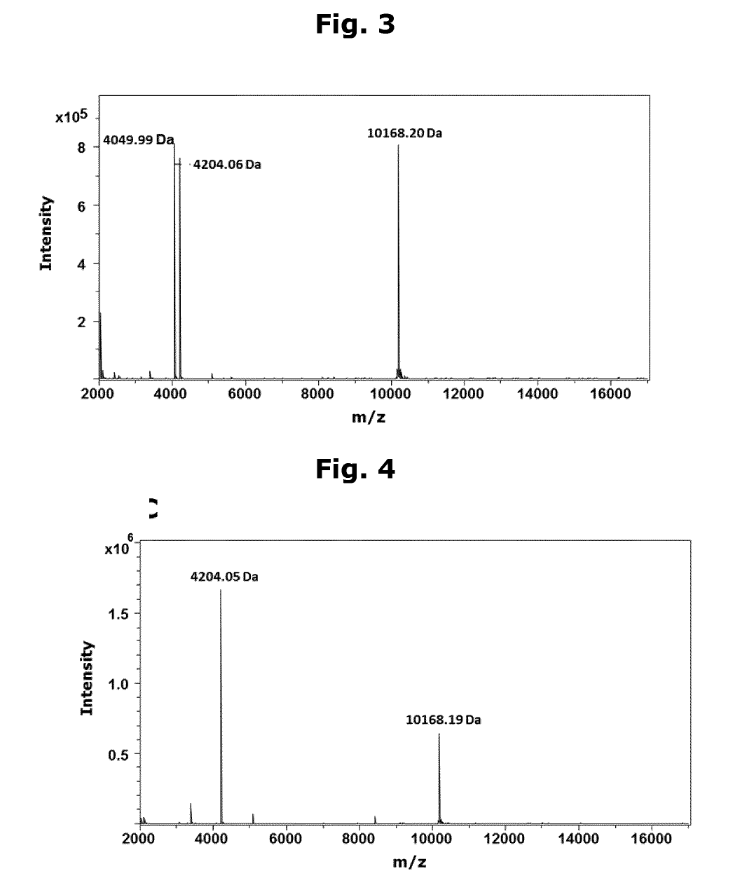

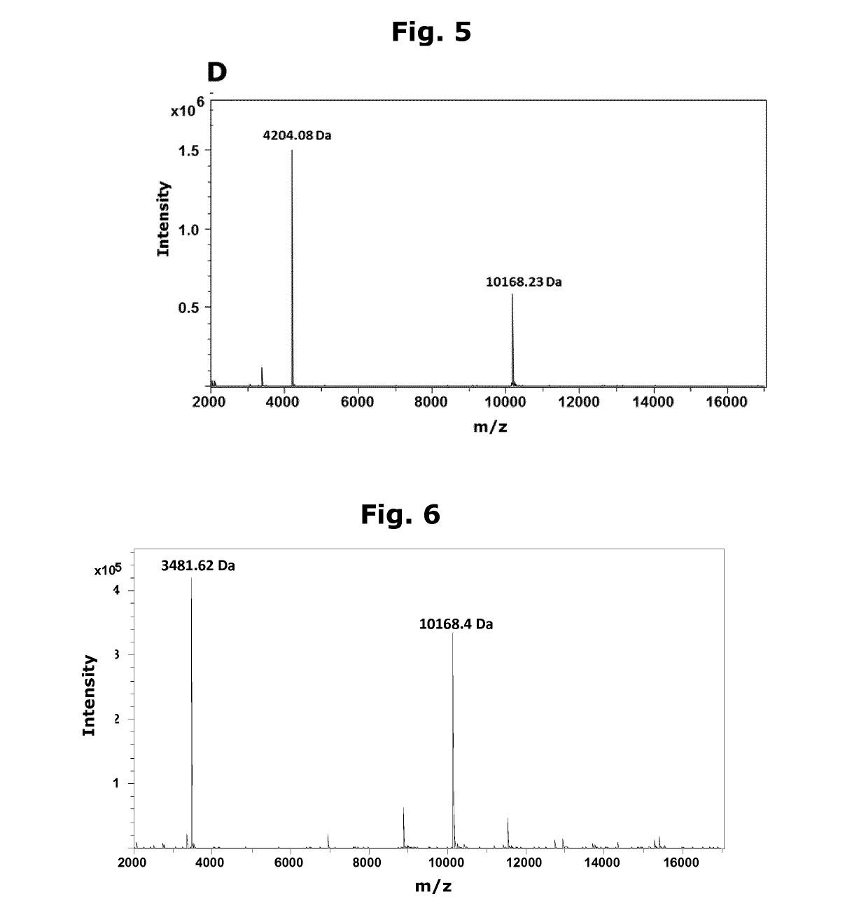

Reaction 1 showed that complete processing of the fusion protein was obtained following enzymatic treatment with an molar enzyme to substrate ratio of 1:20 and 3 hours of incubation at 37° C. (FIG. 2). The predominant determined mass observed was 4049.9 Da, which corresponds to the mass of mature PYY(3-36) (Peak#1) and the released tag (Peak#2). No remaining fusion protein was observed, but a peak with less than 10% of the intensity of Peak#1 was observed which corresponded to GP-PYY(3-36). Reaction 2 shows that a 1:40 enzyme to substrate ratio results in processing of approximately half of the GP-PYY(3-36) into mature PYY(3-36) (FIG. 3). Reaction 3 (FIG. 4) and 4 (FIG. 5) showed that the removal of Gly-Pro from GP-PYY(3-36) observed in Reaction 1 and 2 is specific for the XaaProDAP part of Protease 20986 as the RL9-HRV14 3C protease, which only contains the HRV14 3C domain, is only able to release GP-PYY(3-36).

The experiment shows, that the fully mature PYY(3-36) peptide (4050 Da) can be released by the bifunctional fusion protease, thus enabling the concept of the invention.

### Example 7

### Design of Full-Length Bifunctional Fusion Proteases Comprising Alternative 3C and XaaProDAP Domains from Other Species

In order to demonstrate that other 3C proteases and XaaProDAP enzymes can be fused to obtain functional fusion proteases with the same properties as observed for Protease 20986, 3C protease sequences from Human coxsackievirus B3 (CVB3 3C) or XaaProDAP from Streptococcus suis (S. suis XaaProDAP) were used to replace HRV14 3C and L. lactis XaaProDAP (LLXaaProDAP) sequences and new fusion protease variants were generated. As with the 3C protease sequence from Human Rhino Virus 14 3C, the Human coxsackievirus B3 3C protease sequence also contained a C-terminal Q, which was deleted to obtained CVB3 3C(des183) with the following sequence:

(SEQ ID NO: 23)
GPAFEFAVAMMKRNSSTVKTEYGEFTMLGIYDRWAVLPRHAKPGPTIL
MNDQEVGVLDAKELVDKDGTNLELTLLKLNRNEKFRDIRGFLAKEEVE
VNEAVLAINTSKFPNMYIPVGQVTEYGFLNLGGTPTKRMLMYNFPTRA
GQCGGVLMSTGKVLGIHVGGNGHQGFSAALLKHYFNDE.

A QG site was observed at position Q212-G213 of the S. suis XaaProDAP sequence, which is in proximity to the 3C cleavage site which was determined for the L. Lactis sequence (Q241-G242). A Glu212-Thr213 substitution was introduced to prevent any potential 3C cleavage, thus yielding the following sequence:

(SEQ ID NO: 24)
MRFNQFSFIKKETSVYLQELDTLGFQLIPDASSKTNLETFVRKCHFLT
ANTDFALSNMIAEWDTDLLTFFQSDRELTDQIFYQVAFQLLGFVPGMD
YTDVMDFVEKSNFPIVYGDIIDNLYQLLNTRTKSGNTLIDQLVSDDLI
PEDNHYHFFNGKSMATFSTKNLIREVVYVETPVDTAGTGQTDIVKLSI
LRPHFDGKIPAVITNSPYHETVNDVASDKALHKMEGELAEKQVGTIQV
KQASITKLDLDQRNLPVSPATEKLGHITSYSLNDYFLARGFASLHVSG
VGTLGSTGYMTSGDYQQVEGYKAVIDWLNGRTKAYTDHTRSLEVKADW
ANGKVATTGLSYLGTMSNALATTGVDGLEVIIAEAGISSWYDYYRENG
LVTSPGGYPGEDLDSLTALTYSKSLQAGDFLRNKAAYEKGLAAERAAL
DRTSGDYNQYWHDRNYLLHADRVKCEVVFTHGSQDWNVKPIHVWNMFH
ALPSHIKKHLFFHNGAHVYMNNWQSIDFRESMNALLSQKLLGYENNYQ
LPTVIWQDNSGEQTWTTLDTFGGENETVLPLGTGSQTVANQYTQEDFE
RYGKSYSAFHQDLYAGKANQISIELPVTEGLLLNGQVTLKLRVASSVA
KGLLSAQLLDKGNKKRLAPIPAPKARLSLDNGRYHAQENLVELPYVEM
PQRLVTKGFMNLQNRTDLMTVEEVVPGQWMNLTWKLQPTIYQLKKGDV
LELILYTTDFECTVRDNSQWQIHLDLSQSQLILPH

Three new fusion protease variants were designed comprising the new orthologs of 3C and XaaProDAP using the same His6 fusion partner (SEQ ID NO:13 and the same intervening linker (SEQ ID NO: 12) as described in Example 3. Protease 28994 comprised the L. Lactis XaaProDAP sequence as described for protease 20986 in Example 3A, but the N-terminal HRV14 3C domain was replaced with the 3C domain from Human coxsackievirus B3 (CVB3 3C). Protease 28996 comprised the HRV14 3C sequence as described for Protease 20986 in the N-terminal and the S. suis XaaProDAP sequence in the C-terminal. Protease 28997 is an entirely new fusion protease in which both domains were replaced by other orthologs of 3C and XaaProDAP protease, thus comprising the CVB3 3C sequence in the N-terminal and the S. suis XaaProDAP sequence in the C-terminal of the protease. Plasmid constructs using the pET22b vector backbone and comprising the new fusion proteases were obtained from GenScript. The combination of sequences encoding the designed fusion protease variants are depicted in Table 7.

TABLE 7
pET22b plasmid constructs encoding variants of fusion proteases comprising
combinations of N-terminal HRV14 3C or CVB3 3C and C-terminal L. lactis
XaaProDAP(Q241E, G242T) or S. suis XaaProDAP(Q212E, G213T).

| | | | HRV14 3C | | XaaProDAP | |
|---|---|---|---|---|---|---|
| | | Fusion | domain | Gly-Ser | enzyme | GSS |
| Protease | Product name | partner | (N-term) | linker | (C-term) | extension |
| 28994 | His-CVB3_3C- | SEQ ID | SEQ ID | SEQ ID | SEQ ID NO: 1 | GSS |
| | LLXaaProDAP- | NO: 13 | NO: 23 | NO: 12 | (Q241E, G242T) | |
| | (Q241E, G242T) | | | | | |
| 28996 | His-HRV14_3C- | SEQ ID: | SEQ ID | SEQ ID | SEQ ID NO: 24 | |
| | SSXaaProDAP- | NO: 13 | NO: 11 | NO: 12 | (Q212E, G213T) | |
| | (Q212E, G213T) | | | | | |
| 28997 | His-CVB3, 3C- | SEQ ID | SEQ ID | SEQ ID | SEQ ID NO: 24 | |
| | SSXaaProDAP | NO: 13 | NO: 23 | NO: 12 | (Q212E, G213T) | |
| | (Q212E, G213T) | | | | | |

Small scale expression and IMAC purification of the new fusion protease constructs were conducted as described in Example 1 and showed that all three new proteases yielded soluble and intact fusion proteases comprising the new ortholog sequences of 3C and XaaProDAP.

Intact mass was determined by LC-MS analysis of the IMAC purified fusion protease variants as described in Example 1 and results confirmed the observations from SDS-PAGE. Protease 28994, 28996 and 28997 had determined masses of 107797.8 Da, 107687.2 Da and 107964.2 Da, respectively, which are in excellent agreement with the calculated masses 107798.1 Da, 107687.4 Da and 107964.8 Da, respectively. Thus, as observed with Protease 20986, the new proteases were not significantly truncated or degraded, as the predominant detected masses corresponding to the calculated mass for the full-length fusion proteases. Hence, all the Proteases 20986, 28994, 28996 and 28997 have substantially no self-cleavage activity able to deteriorate at least one of the two constituent proteolytic activities. In conclusion, the concept of preparing functional 3C/XaaProDAP fusion proteases was further demonstrated for the present invention using other orthologs of the picornaviral 3C and XaaProDAP enzymes with highly different aa sequences.

### Example 8

### Scaling Up Expression and Purification of Protease 28994, 28996 and 28997 Comprising New Domains of 3C and XaaProDAP

Expression of Protease 28994, 28996 and 28997 was done as described in Example 4 using BL21(DE3) as expression host. Purification was done essentially as described in Example 4 utilizing a IMAC step for capture followed by a gel filtration step. Protease 28994, 28996 and 28997 were all successfully purified by a two step protocol as described in Example 4. The purity of the enzymes were estimated to be at least 90% as judged by inspection of SDS-PAGE gels and by evaluation of UV215 nm profiles from RP separation HPLC during LC-MS analysis. MS analysis was done as described in Example 1 and showed that protease 28994 had an estimated mass of 107797.8 Da in close agreement with the expected mass (110798.1 Da, average isotopic mass). Protease 28996 had a mass of 107686.9 Da in close agreement with the expected mass (107687.4 Da, average isotopic mass) and Protease 28997 had a determined mass of 107964.8 Da in agreement with the expected mass (107964.8, average isotopic mass). UV280 absorbance measurement was used to determine the concentration of the fusion proteins (NanoDrop).

### Example 9

### Enzymatic Reactions with Protease 20986, 28994, 28996 and 28997

Enzymatic reaction were setup in reaction volumes of 30 μl using 1×PBS, pH 7.4 as enzyme reaction buffer. The model protein substrates used for evaluation of cleavage specificity comprised fusion proteins, which following correct processing by the enzymes should yield human PYY(3-36)(SEQ ID NO: 18), wt Glucagon (SEQ ID NO: 19) and GLP-1(7-37, K34R)(SEQ ID NO: 20). The concentration of model protein substrates was adjusted to 0.5 mg/ml with 1×PBS, pH 7.4 as described in Example 6. Variations of reaction conditions were evaluated both in terms of enzyme to substrate ratios as well as duration and temperature of the enzymatic reactions. Controls without enzyme (1×PBS pH 7.4) or with RL9-HRV14 3C (SEQ ID NO. 21) was included. Reactions were stopped by addition of >0.5 M AcOH at the end of the experiment. LC-MS analysis of enzymatic reactions was done using the conditions and general settings as described in Example 6

RL27_EVLFQGP_PYY(3-36) as Model Protein Substrate

Incubations of Protease 28994, 28996 and 28997 with RL27-EVLFQGP-PYY(3-36) substrate was setup using molar enzyme to substrate ratios of 1:20 or 1:100, respectively, and the reactions were carried out for 3 hours at 37° C. (as depicted in Table 8). Analysis of intact masses by LC-MS showed that Protease 28994, 28996 and 28997 were able to process the RL27_EVLFQGP_PYY(3-36) completely to mature PYY(3-36) (SEQ ID NO: 18) following 3 hours incubation at 37° C. (as observed for 20986 in Example 6), when using an enzyme to substrate molar ratio of 1:20. At 1:100 enzyme to substrate ratio, lower amounts of PYY(3-36) was detected as well as GP-PYY(3-36) (Reaction 6, 8 and 10) and the reaction was not always completed as intact fusion protein was detected. At a ratio of 1:100, Protease 28996 and 28997 provided the most efficient cleavage with lowest amount of remaining GP-PYY(3-36) with relative intensities of ˜25% or ˜50% the intensity of the mature PYY(3-36) peaks, respectively. A control with RL9-HRV14 3C (SEQ ID NO. 21) only yielded GP_PYY(3-36) peaks, showing that XaaProDAP domains are responsible for completing the reaction to yield the native N-terminal of PYY(3-36) and no addition of enzyme only yielded the unprocessed fusion protein. The experiment shows that different fusion protease variants combining 3C proteases from Human Rhino virus or Human Cocksakie virus with XaaProDAP from L. lactis or S. suis can be successfully used to process RL27_EVLFQGP_PYY(3-36) into mature PYY(3-36) with Ile being the correct N-terminal amino acid residue.

TABLE 8
Enzymatic reactions using Protease 28994, 28996 and 28997 from Example 8 and
RL27_EVLFQGP_PYY(3-36) as substrate, all incubated for 3 hours at 37° C.
Experimentally determined predominant peaks detected in deconvoluted mass spectra
of reactions 5-10 are indicated.

| | | | | Determined | Calculated | |
|---|---|---|---|---|---|---|
| Reaction | | Molar | Predominant | mass | mass | |
| number | Enzyme | ratio | detected peaks | (Dalton) | (Dalton) | Corresponds to |
| Reaction 5 | Protease | 1:20 | Peak #1 | 4050.09 | 4050.1 | PYY(3-36) |
| | 28994 | | | | | (SEQ ID NO: 18) |
| | | | Peak #2 | 10168.47 | 10168.4 | RL27 tag |
| Reaction 6 | Protease | 1:100 | Peak #1 | 4050.07 | 4050.1 | PYY(3-36) |
| | 28994 | | | | | (SEQ ID NO: 18) |
| | | | Peak#2 | 10168.42 | 10168.4 | RL27 tag |
| | | | Peak#3 | 4204.14 | 4204.1 | GP-PYY(3-36) |
| | | | Peak#4 | 14354.54 | 14354.5 | RL27_EVLFQGP_ |
| | | | | | | PYY(3-36) |
| Reaction 7 | Protease | 1:20 | Peak #1 | 4050.09 | 4050.1 | PYY(3-36) |
| | 28996 | | | | | (SEQ ID NO: 18) |
| | | | Peak #2 | 10168.46 | 10168.4 | RL27 tag |
| Reaction 8 | Protease | 1:100 | Peak #1 | 4050.09 | 4050.1 | PYY(3-36) |
| | 28996 | | | | | (SEQ ID NO: 18) |
| | | | Peak#2 | 10168.47 | 10168.4 | RL27 tag |
| | | | Peak#3 | 4204.16 | 4204.1 | GP-PYY(3-36) |
| Reaction 9 | Protease2 | 1:20 | Peak #1 | 4050.10 | 4050.1 | PYY(3-36) |
| | 28997 | | | | | (SEQ ID NO: 18) |
| | | | Peak #2 | 10168.49 | 10168.4 | RL27 tag |
| Reaction 10 | Protease | 1:100 | Peak #1 | 4050.09 | 4050.1 | PYY(3-36) |
| | 28997 | | | | | (SEQ ID NO: 18) |
| | | | Peak#2 | 10168.47 | 10168.4 | RL27 tag |
| | | | Peak#3 | 4204.16 | 4204.1 | GP-PYY(3-36) |
| | | | Peak#4 | 14354.62 | 14354.5 | RL27_EVLFQGP_ |
| | | | | | Da | PYY(3-36) |

RL27_EVLFQGP_Glucagon as Model Protein Substrate

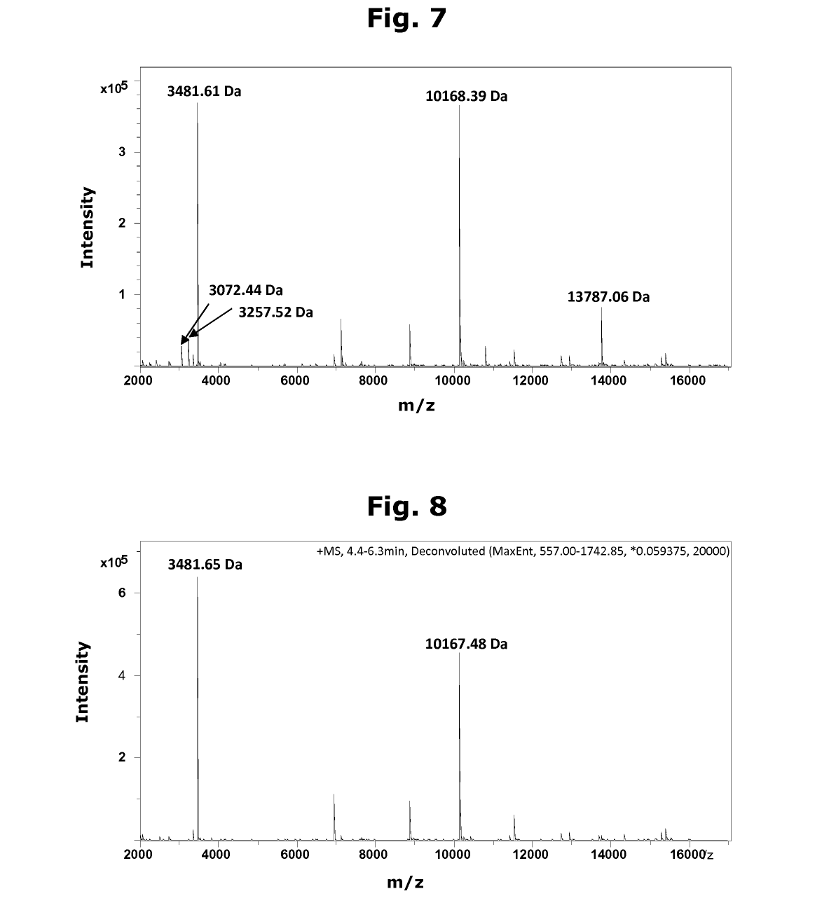

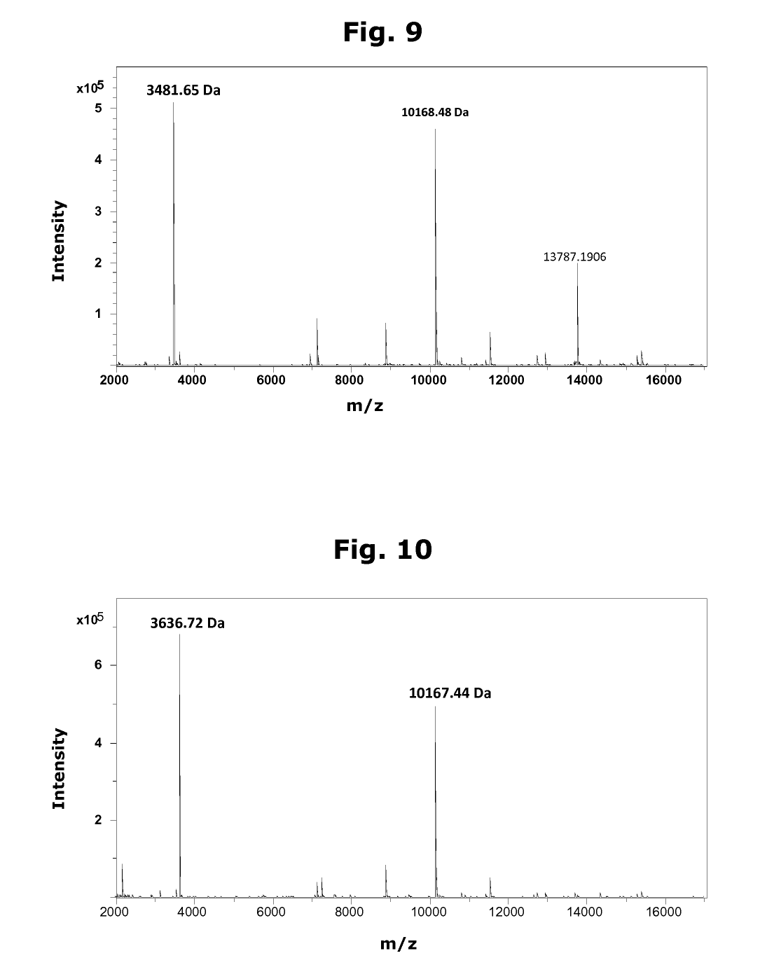

Incubations of Protease 20986, 28994, 28996 and 28997 with RL27-EVLFQGP-Glucagon substrate was setup as described above. Analysis of intact masses by LC-MS showed that Protease 20986, 28994, 28996 and 28997 were all able to process the RL27_EVLFQGP_Glucagon to mature Glucagon with differences observed in overall efficiency and specificity using 1:100 or 1:500 enzyme to substrate ratio with either 4° C. or 37° C. as incubation temperatures (FIG. 6-9). For Protease 20986, 28996, 1:500 enzyme to substrate ratio and incubation temperatures at 4° C. (Table 9, Reaction 11 and 16)) gave the most optimal cleavage conditions with complete processing of the fusion protein and no significant unspecific cleavage (FIGS. 6 and 8). The determined mass of released Glucagon was in agreement with the calculated mass of 3482.8 Da for human wt Glucagon (Peak #1). Protease 28994 and 28997 was less efficient and did not completely process all fusion protein at the tested conditions and for Protease 28994, peaks with low intensity (Peak #3 and #4) indicated very limited unspecific cleavage (Table 9, Reaction 13 (FIG. 7) 14 and 17 (FIG. 9)). A control with RL9-HRV14 3C (SEQ ID NO. 21) only yielded GP_Glucagon (Reaction 18, FIG. 10), showing that XaaProDAP domains are responsible for completing the reaction to yield the native N-terminal Histidine in Glucagon (SEQ ID NO: 19). No addition of enzyme only yielded the unprocessed fusion protein with a determined mass in agreement with the calculated mass of 13787.1 Da for RL27_EVLFQGP_Glucagon without the Initiator Methionine. This shows that different fusion protease variants combining picornaviral 3C proteases from Human Rhino virus or Human cocksakie virus with XaaProDAP from L. lactis or S. suis can be successfully optimized to process the RL27_EVLFQGP_Glucagon into mature Glucagon with His as the correct N-terminal amino acid residue and with no or very limited generation of fusion protein related impurities

TABLE 9
Enzymatic reactions using Protease 20986, 28994, 28996 and 28997,
and RL27_EVLFQGP_Glucagon as substrate at 4° C. overnight
incubations. Experimentally determined predominant peaks detected
in deconvoluted mass spectra of reactions 11-18 are indicated.

| | | | Pre- | Determined | Calculated | Corre- |
|---|---|---|---|---|---|---|
| Reaction | | Molar | dominant | mass | mass | sponds |
| number | Enzyme | ratio | peaks | (Dalton) | (Dalton) | to |
| Reaction | 20986 | 1:100 | Peak#1 | 3482.61 | 3482.8 | Glucagon |
| 11 | | | | | | (SEQ ID |
| | | | | | | NO: 19) |
| | | | Peak#2 | 10168.37 | 10168.4 | RL27 tag |
| Reaction | 20986 | 1:500 | Peak#1 | 3481.62 | 3482.8 | Glucagon |
| 12 | | | | | | (SEQ ID |
| | | | | | | NO: 19) |
| | | | Peak#2 | 10168.4 | 10168.4 | RL27 tag |
| Reaction | 28994 | 1:100 | Peak#1 | 3481.61 | 3482.8 | Glucagon |
| 13 | | | | | | (SEQ ID |
| | | | | | | NO: 19) |
| | | | Peak#2 | 10168.39 | 10168.4 | RL27 tag |
| | | | Peak#3 | 3257.52 | 3258.6 | Glucagon |
| | | | | | | (3-29) |
| | | | Peak#4 | 3072.44 | 3073,4 | Glucagon |
| | | | | | | (5-29) |
| | | | Peak#5 | 13787.06 | 13787.1 | RL27_ |
| | | | | | | EVLFQP- |
| | | | | | | Glucagon |
| Reaction | 28994 | 1:500 | Peak#1 | 3481.62 | 3482.8 | Glucagon |
| 14 | | | | | | (SEQ ID |
| | | | | | | NO: 19) |
| | | | Peak#2 | 10168.41 | 10168.4 | RL27 tag |
| | | | Peak#3 | 13787.09 | 13787.1 | RL27_ |
| | | | | | | EVLFQP- |
| | | | | | | Glucagon |
| Reaction | 28996 | 1:100 | Peak#1 | 3481.63 | 3482.8 | Glucagon |
| 15 | | | | | | (SEQ ID |
| | | | | | | NO: 19) |
| | | | Peak#2 | 10168.46 | 10168.4 | RL27 tag |
| Reaction | 28996 | 1:500 | Peak#1 | 3481.65 | 3482.8 | Glucagon |
| 16 | | | | | | (SEQ ID |
| | | | | | | NO: 19) |
| | | | Peak#2 | 10167.48 | 10168.4 | RL27 tag |
| Reaction | 28997 | 1:500 | Peak#1 | 3481.65 | 3482.8 | Glucagon |
| 17 | | | | | | (SEQ ID |
| | | | | | | NO: 19) |
| | | | Peak#2 | 10168.48 | 10168.4 | RL27 tag |
| | | | Peak#3 | 13787.19 | 13787.1 | RL27_ |
| | | | | | | EVLFQP- |
| | | | | | | Glucacon |
| Reaction | RL9- | 1:20 | Peak#1 | 3636.72 | 3636.7 | GP- |
| 18 | HRV14 | | | | | Glucagon |
| | 3C | | | | | |
| | | | Peak #2 | 10167.44 | 10168.4 | RL27 tag |

RL27_EVLFQGP_GLP-1(7-37,K34R) as Model Protein Substrate.

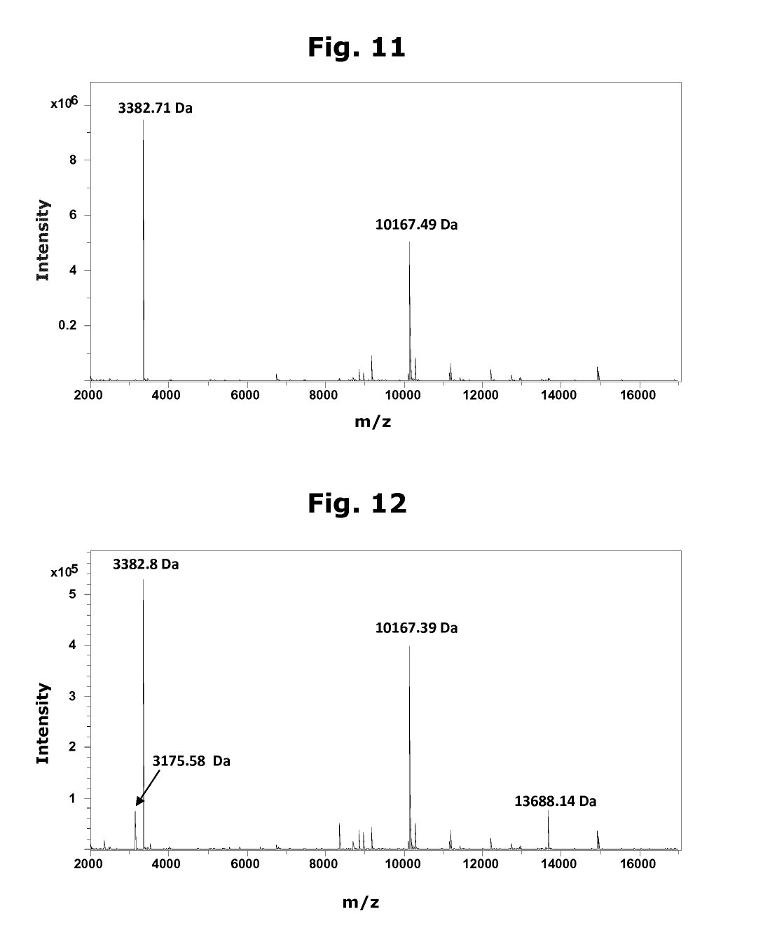

Incubations of Protease 20986, 28994, 28996 and 28997 with RL27_EVLFQGP_GLP-1(7-37,K34R) substrate was setup as described above. Analysis of intact masses by LC-MS showed that Protease 20986, 28994, 28996 and 28997 were all able to fully process the RL27_EVLFQGP_GLP-1 in to mature GLP-1(7-37,K34R) with a determined molecular mass corresponding to the calculated mass of 3382.7 Da (Table 10, FIG. 11-14). Minor differences were observed in overall efficiency and specificity using 1:100 or 1:500 enzyme to substrate ratio with either 4° C. or 37° C. as incubation temperatures. Unspecific fragments observed were predominantly GLP-1(9-37, K34R) (Calculated mass of 3174.6 Da), where an additional dipeptide was removed from the GLP-1 sequence. In this experimental setting, the most optimal cleavage conditions were obtained at 4° C. with complete processing of the fusion protein and very limited or no unspecific cleavage. Protease 28994 was less efficient (Reaction 21 (FIG. 12) and 22, Table 10) as remaining fusion protein was observed following incubation. Protease 28996, gave complete cleavage of fusion protein and release of mature GLP-1(7-37, K34R) with no observed unspecific cleavage using 3 h at 37° C. (not shown).

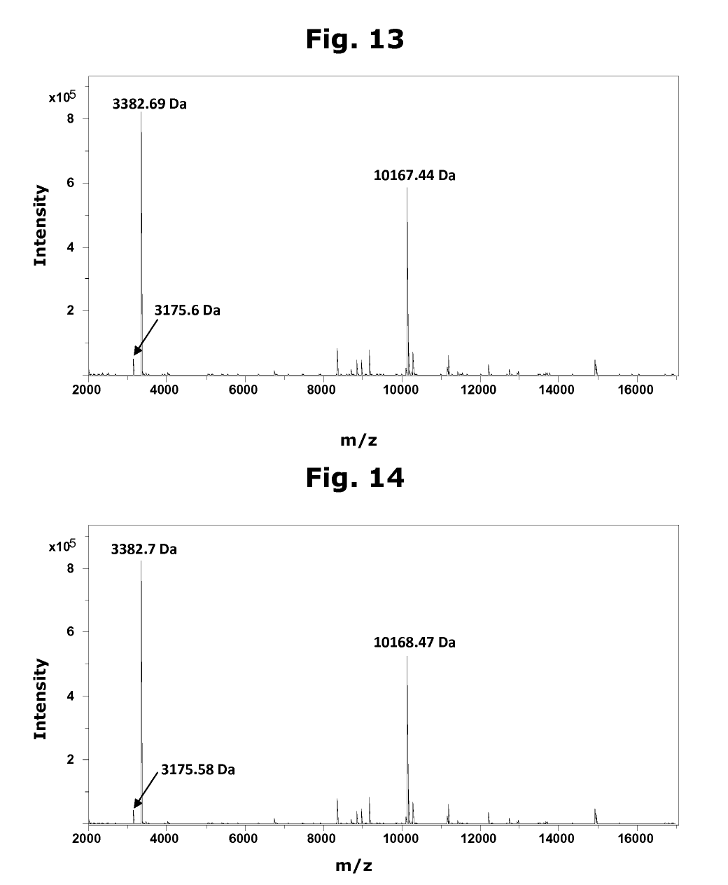

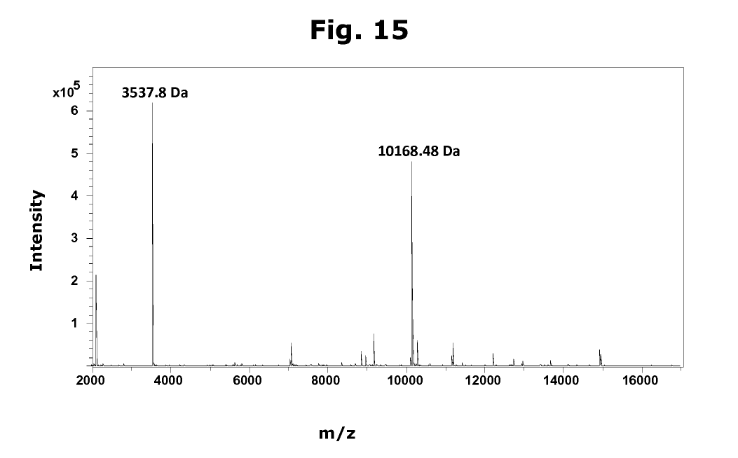

The most efficient reactions were obtained with Protease 20986 which had optimal cleavage conditions using 1:500 enzyme to substrate ratio with overnight incubation at 4° C., without detectable contributions of fragments derived from unspecific or incomplete processing (Reaction 20, FIG. 11). Similar results were obtained with Protease 28996 and 28997 (Reaction 23 (FIG. 13) & 25 (FIG. 14)), which almost exclusively yielded fully processed mature GLP-1(7-37,K34R) using 1:100 enzyme to substrate ratio and incubation at 4° C. overnight, whereas small, but detectable amount of unprocessed GP-GLP-1(7-37,K34R) (˜10% of intensity of mature peak) could be detected after incubation with 1:500 ratio (Reaction 24 & 26)). A control with RL9-HRV14 3C (SEQ ID NO:21) only yielded GP_GLP-1(7-37,K34R) as expected (Reaction 27, FIG. 15), showing that XaaProDAP enzyme domains of the fusion proteases are responsible for providing the native N-terminal Histidine in GLP-1(7-37, K34R). No addition of enzyme only yielded the unprocessed fusion protein with a determined mass in agreement with the calculated mass of 13688.1 Da, corresponding to RL27_EVLFQGP_GLP-1(7-37,K34R) without the initiator Methionine. Thus, different fusion protease variants combining picornaviral 3C proteases from Human Rhino virus or Human cocksakie virus with XaaProDAP from L. lactis or S. suis can be optimized to process the RL27_EVLFQGP_GLP-1(7-37,K34R) into mature GLP-1(7-37,K34R) (SEQ ID NO:20) with His as the correct N-terminal aa residue and with no or very limited generation of fusion protein related impurities.

TABLE 10
Enzymatic reactions using Proteases 20986, 28994, 28996 and 28997, and
RL27_EVLFQGP_GLP-1(7-37, K34R) as substrate at 4° C., overnight incubations.
Experimentally determined predominant peaks detected in deconvoluted mass
spectra of reactions 19-27 are indicated.

| | | | | Determined | Calculated | |
|---|---|---|---|---|---|---|
| Reaction | | Molar | Predominant | mass | mass | |
| number | Enzyme | ratio | peaks | (Dalton) | (Dalton) | Corresponds to |
| Reaction 19 | 20986 | 1:100 | Peak#1 | 3174.6 | 3174.6 | GLP-1(9-37, K34R) |
| | | | Peak#2 | 3382.7 | 3382.7 | GLP-1(7-37, K34R) |
| | | | | | | (SEQ ID NO: 20) |
| | | | Peak#3 | 10168.46 | 10168.4 | RL27 tag |
| Reaction 20 | 20986 | 1:500 | Peak#1 | 3382.71 | 3382.7 | GLP-1(7-37, K34R) |
| | | | | | | (SEQ ID NO: 20) |
| | | | Peak#2 | 10167.49 | 10168.4 | RL27 tag |
| Reaction 21 | 28994 | 1:100 | Peak#1 | 3175.58 | 3174.6 | GLP-1(9-37, K34R) |
| | | | Peak#2 | 3382.68 | 3382.7 | GLP-1(7-37, K34R) |
| | | | | | | (SEQ ID NO: 20) |
| | | | Peak#3 | 10167.39 | 10168.4 | RL27 tag |
| | | | Peak#4 | 13688.14 | 13688.1 | RL27_EVLFQGP_GLP- |
| | | | | | | 1(7-37, K34R) |
| Reaction 22 | 28994 | 1:500 | Peak#1 | 3382.67 | 3382.7 | GLP-1(7-37, K34R) |
| | | | | | | (SEQ ID NO: 20) |
| | | | Peak#2 | 10168.4 | 10168.4 | RL27 tag |
| | | | Peak#3 | 13688.15 | 13688.1 | RL27_EVLFQGP_GLP- |
| | | | | | | 1(7-37, K34R) |
| Reaction 23 | 28996 | 1:100 | Peak#1 | 3174.6 | 3174.6 | GLP-1(9-37, K34R) |
| | | | Peak#2 | 3382.69 | 3382.7 | GLP-1(7-37, K34R) |
| | | | | | | (SEQ ID NO: 20) |
| | | | Peak#3 | 10167.44 | 10168.4 | RL27 tag |
| Reaction 24 | 28996 | 1:500 | Peak#1 | 3382.7 | 3382.7 | GLP-1(7-37, K34R) |
| | | | | | | (SEQ ID NO: 20) |
| | | | Peak#2 | 10168.48 | 10168.4 | RL27 tag |
| | | | Peak#3 | 3537.77 | 3537.7 | GP-GLP-1(7-37, K34R) |
| Reaction 25 | 28997 | 1:100 | Peak#1 | 3382.7 | 3382.7 | GLP-1(7-37, K34R) |
| | | | | | | (SEQ ID NO: 20) |
| | | | Peak#2 | 10168.47 | 10168.4 | RL27 tag |
| Reaction 26 | 28997 | 1:500 | Peak#1 | 3382.71 | 3382.7 | GLP-1(7-37, K34R) |
| | | | | | | (SEQ ID NO: 20) |
| | | | Peak#2 | 10167.49 | 10168.4 | RL27 tag |
| | | | Peak#3 | 3537.78 | 3537.7 | GP-GLP-1(7-37, K34R) |
| Reaction 27 | RL9 | | Peak#1 | 3537.78 | 3537.7 | GP-GLP-1(7-37, K34R) |
| | HRV14 | | | | | |
| | 3C | | | | | |
| | | | Peak#2 | 10168.48 | 10168.4 | RL27 tag |

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A bifunctional fusion enzyme comprising the catalytic domains of a picornaviral 3C protease and a XaaProDAP.

2. The bifunctional fusion protease according to claim 1 comprising a protein of the formula:
X—Y—Z (I) or
Z—Y—X (II)
wherein
X is a picornaviral 3C protease or a functional variant thereof;
Y is an optional linker;
Z is a Xaa-Pro-dipeptidyl aminopeptidase (XaaProDAP) or a functional variant thereof;
wherein said fusion protease has substantially no self-cleavage activity able to deteriorate at least one of the two proteolytic activities.

3. The bifunctional fusion protease according to claim 2 comprising a protein of formula (I), wherein said picornaviral 3C protease or a functional variant thereof is in the N-terminal part of said bifunctional fusion protease.

4. The bifunctional fusion protease according to claim 2, wherein X is a human Rhinovirus 3C protease or a functional variant thereof.

5. The bifunctional fusion protease according to claim 2, wherein X comprises SEQ ID NO: 2, or a functional variant thereof.

6. The bifunctional fusion protease according to claim 2, wherein Z is an E.C. 3.4.14.11 enzyme or a functional variant thereof.

7. The bifunctional fusion protease according to claim 6, wherein Z is an enzyme from a lactic acid bacterium or a functional variant thereof.

8. The bifunctional fusion protease according to claim 2, wherein Z is SEQ ID NO: 1 or a functional variant thereof.

9. The bifunctional fusion protease according to claim 2, wherein Z is an enzyme from Streptococcus spp. or a functional variant thereof.

10. The bifunctional fusion protease according to claim 9 wherein Z is SEQ ID NO: 24 or a functional variant thereof.

11. The bifunctional fusion protease according to claim 2, wherein said functional variant comprises from 1-15 amino acid substitutions, deletions or additions relative to the corresponding naturally occurring protein or naturally occurring sub-sequence of a protein.

12. The bifunctional fusion protease according to claim 2, comprising a linker Y.

13. The bifunctional fusion protease according to claim 1, further comprising a tag protein attached to the N-terminal.

14. A method for preparing a bifunctional fusion protease according to claim 1, comprising recombinantly expressing a protein comprising the bifunctional fusion protease in a host cell and subsequently isolating the bifunctional fusion protease.

15. A method for removing an N-terminal peptide or protein from a larger peptide or protein comprising the use of the bifunctional fusion protease according to claim 1.
